(19) United States
(12) Patent Application Publication
(10) Pub. No.: US 2023/0095696 A1
(43) Pub. Date: Mar. 30, 2023

(54) OLIGOMERIC COMPOUNDS COMPRISING BACKBONE CONSTRAINED MACROCYCLES

(71) Applicant: Ionis Pharmaceuticals, Inc., Carlsbad, CA (US)

(72) Inventors: Juan Carlos Salinas Hernandez, Montreal (CA); Stephen Hanessian, Beaconsfield (CA); Eric E. Swayze, Encinitas, CA (US); Punit P. Seth, Carlsbad, CA (US); Jenny Lorena Rico Duque, Montreal (CA); Graeme C. Freestone, La Jolla, CA (US); Barbara Lugato, Montreal (CA); Rajasekaran Tamiselvan, Montreal (CA)

(73) Assignee: Ionis Pharmaceuticals, Inc., Carlsbad, CA (US)

(21) Appl. No.: 17/822,202

(22) Filed: Aug. 25, 2022

Related U.S. Application Data
(60) Provisional application No. 63/237,082, filed on Aug. 25, 2021.

(51) Int. Cl. C07H 21/00; C12N 15/113

(52) U.S. Cl. CPC C07H 21/00; C12N 15/113; C12N 2310/11

(57) ABSTRACT

The present disclosure provides a trinucleotide comprising the formula below or an oligomeric compound comprising the formula below:

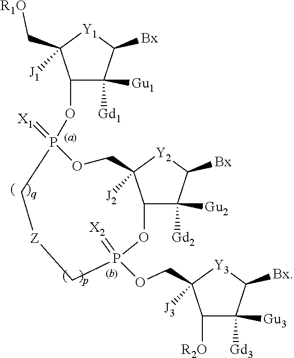

### SEQUENCE LISTING

The present application is being filed along with a Sequence Listing in electronic format. The Sequence Listing is provided as a file entitled CHEM0100USSEQ.xml, created on Aug. 23, 2022 which is 7.83 Kb in size. The information in the electronic format of the sequence listing is incorporated herein by reference in its entirety.

### FIELD OF THE INVENTION

The present disclosure provides backbone constrained nucleic acids and backbone constrained antisense oligomeric compounds prepared therefrom.

### BACKGROUND OF THE INVENTION

The principle behind antisense technology is that an antisense compound hybridizes to a target nucleic acid and modulates the amount, activity, and/or function of the target nucleic acid. For example, in certain instances, antisense compounds result in altered transcription or translation of a target. Such modulation of expression can be achieved by, for example, target RNA degradation or occupancy-based inhibition. An example of modulation of RNA target function by degradation is RNase H-based degradation of the target RNA upon hybridization with a DNA-like antisense compound.

Another example of modulation of gene expression by target degradation is RNA interference (RNAi). RNAi refers to antisense-mediated gene silencing through a mechanism that utilizes the RNA-induced silencing complex (RISC). An additional example of modulation of RNA target function is by an occupancy-based mechanism such as is employed naturally by microRNA. MicroRNAs are small non-coding RNAs that regulate the expression of protein-coding RNAs. The binding of an antisense compound to a microRNA prevents that microRNA from binding to its messenger RNA targets, and thus interferes with the function of the microRNA. MicroRNA mimics can enhance native microRNA function. Certain antisense compounds alter splicing of pre-mRNA. Another example of modulation of gene expression is the use of antisense compounds in a CRISPR system. Regardless of the specific mechanism, sequence-specificity makes antisense compounds attractive as tools for target validation and gene functionalization, as well as therapeutics to selectively modulate the expression of genes involved in the pathogenesis of disease.

Antisense technology is an effective means for reducing the expression of one or more specific gene products and can therefore prove to be uniquely useful in a number of therapeutic, diagnostic, and research applications Chemically modified nucleosides and chemically modified internucleoside linkages are routinely used for incorporation into oligomeric compounds comprising modified oligonucleotides to enhance one or more properties, such as nuclease resistance, pharmacokinetics or affinity for a target RNA.

### BRIEF SUMMARY OF THE INVENTION

Disclosed herein are novel backbone constrained nucleoside units, comprising three linked nucleosides, and oligomeric compounds comprising modified oligonucleotides prepared therefrom. The present disclosure provides for the joining of two adjacent nucleosides within a modified oligonucleotide by a carbon or heteroatom-containing chain linking two adjacent phosphorus atoms of the backbone to form a macrocycle. To facilitate synthesis of a modified oligonucleotide or oligomeric compound comprising such a backbone constrained pair of linked nucleosides, the two nucleosides connected through the backbone to form a macrocycle are further attached to a third nucleoside to form the backbone constrained nucleoside unit.

In certain embodiments, the oligomeric compounds provided herein comprise modified oligonucleotides that hybridize to a portion of a target nucleic acid and result in modulation of the amount or activity of the target nucleic acid.

In certain embodiments, the present disclosure provides backbone constrained nucleoside units. In certain embodiments, the present disclosure provides a backbone constrained nucleoside unit comprising the formula below:

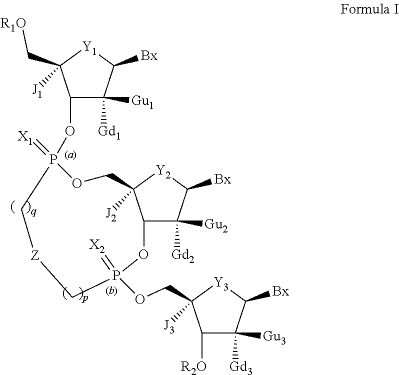

wherein each Bx is, independently, a heterocyclic base moiety;
R₁ is H, a hydroxyl protecting group or a conjugate group;
R₂ is H, a hydroxyl protecting group, a conjugate group, or a reactive phosphorous group;
(a) and (b) are the stereochemistry at the phosphate and are independently selected from (R), (S), and (R,S);
p is from 1 to 7;
q is from 1 to 7;
and wherein p+q is greater than or equal to 4;
Z is CH₂ or O;
each X is, independently, O or S;
each Y is, independently, O or S;
either J₁ and G_{d1} form a J₁ and G_{d1} bridge and Gᵤ₁ is H, or J₁ is H and G_{d1} and Gᵤ₁ are independently selected from H, OH, halogen or O—[C(R₃)(R₄)]ₙ—[(C═O)ₘ—X^{G}]ⱼ—R₅;
either J₂ and G_{d2} form a J₂ and G_{d2} bridge and Gᵤ₁ is H, or J₂ is H and G_{d2} and Gᵤ₂ are independently selected from H, OH, halogen or O—[C(R₃)(R₄)]ₙ—[(C═O)ₘ—X^{G}]ⱼ—R₅;
either J₃ and G_{d3} form a J₃ and G_{d3} bridge and Gᵤ₁ is H, or J₃ is H and G_{d3} and Gᵤ₃ are independently selected from H, OH, halogen or O—[C(R₃)(R₄)]ₙ—[(C═O)ₘ—X^{G}]ⱼ—R₅;
wherein each J to G_{d} bridge has a formula independently selected from —CH(CH₃)—O— or —(CH₂)ₖ—O—,
   wherein k is from 1 to 3;
   each R₃ and R₄ is, independently, H, halogen, C₁-C₆ alkyl or substituted C₁-C₆ alkyl;
each X^{G} is O, S or N(E₁);
R₅ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(═X₂)J₁, OC(═X₂)N(J₁)(J₂) and C(=Q₂)N(J₁)(J₂);
Q₂ is O, S or NJ₃;
each J₁, J₂ and J₃ is, independently, H or C₁-C₆ alkyl.

In certain embodiments, the present disclosure provides a compound comprising a modified oligonucleotide comprising 8 to 40 linked nucleosides, wherein the modified oligonucleotide comprises a region having the formula:

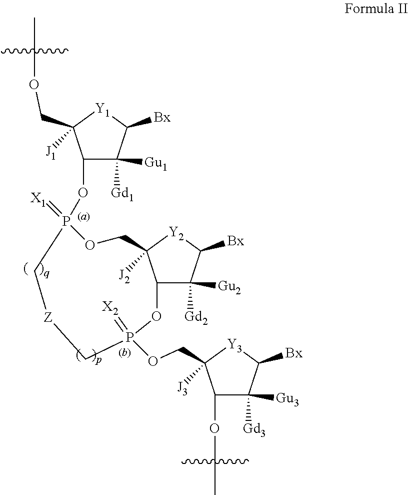

wherein each Bx is, independently, a heterocyclic base moiety;
(a) and (b) are the stereochemistry at the phosphate and are independently selected from (R), (S), and (R,S);
p is from 1 to 7;
q is from 1 to 7;
and wherein p+q is greater than or equal to 4;
Z is CH₂ or O;
each X is, independently, O or S;
each Y is, independently, O or S;
either J₁ and G_{d1} form a J₁ and G_{d1} bridge and Gᵤ₁ is H, or J₁ is H and G_{d1} and Gᵤ₁ are independently selected from H, OH, halogen or O—[C(R₃)(R₄)]ₙ—[(C═O)ₘ—X^{G}]ⱼ—R₅;
either J₂ and G_{d2} form a J₂ and G_{d2} bridge and Gᵤ₁ is H, or J₂ is H and G_{d2} and Gᵤ₂ are independently selected from H, OH, halogen or O—[C(R₃)(R₄)]ₙ—[(C═O)ₘ—X^{G}]ⱼ—R₅;
either J₃ and G_{d3} form a J₃ and G_{d3} bridge and Gᵤ₁ is H, or J₃ is H and G_{d3} and Gᵤ₃ are independently selected from H, OH, halogen or O—[C(R₃)(R₄)]ₙ—[(C═O)ₘ—X^{G}]ⱼ—R₅;
wherein each J to G_{d} bridge has a formula independently selected from —CH(CH₃)—O— or —(CH₂)ₖ—O—, wherein k is from 1 to 3;
   each R₃ and R₄ is, independently, H, halogen, C₁-C₆ alkyl or substituted C₁-C₆ alkyl;
each X^{G} is O, S or N(E₁);
R₅ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(═X₂)J₁, OC(═X₂)N(J₁)(J₂) and C(=Q₂)N(J₁)(J₂);
Q₂ is O, S or NJ₃;
each J₁, J₂ and J₃ is, independently, H or C₁-C₆ alkyl.

### DETAILED DESCRIPTION OF THE INVENTION

Provided herein are novel backbone constrained nucleoside units, comprising three linked nucleosides, and oligomeric compounds comprising modified oligonucleotides prepared therefrom. The novel backbone constrained nucleoside units are expected to be useful for enhancing one or more properties of the oligomeric compounds they are incorporated into, such as, for example nuclease resistance. In certain embodiments, the oligomeric compounds provided herein comprise a modified oligonucleotide that can hybridize to a portion of a target RNA, modifying the function of the target RNA. In certain embodiments, the modified oligonucleotide is an antisense modified oligonucleotide.

In certain embodiments, the novel backbone constrained nucleoside units provided herein are incorporated into oligomeric compounds comprising antisense modified oligonucleotides which are used to reduce target RNA, such as messenger RNA, in vitro and in vivo. The reduction of target RNA can be effected via numerous pathways with a resultant modulation of gene expression. Such modulation can provide direct or indirect increase or decrease in a particular target (nucleic acid or protein). Such pathways include, for example, the steric blocking of transcription or translation, as well as enzyme-mediated cleavage of mRNA using single stranded oligomeric compounds or duplexes comprising two oligomeric compounds. The oligomeric compounds provided herein are also expected to be useful as primers and probes in diagnostic applications. In certain embodiments, oligomeric compounds comprising at least one region of backbone constrained nucleoside units as provided herein are expected to be useful as aptamers, which are oligomeric compounds capable of binding to selected proteins in an in vivo setting.

Incorporation of one or more regions of backbone constrained nucleoside units, as provided herein, into an oligomeric compound is expected to alter one or more properties of the resulting oligomeric compound. Such properties include without limitation stability, nuclease resistance, binding affinity, specificity, absorption, cellular distribution, cellular uptake, charge, pharmacodynamics and pharmacokinetics.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, the term “alkyl” refers to a saturated straight or branched hydrocarbon radical containing up to twenty four carbon atoms. Examples of alkyl groups include without limitation, methyl, ethyl, propyl, butyl, isopropyl, n-hexyl, octyl, decyl, dodecyl and the like. Alkyl groups typically include from 1 to about 24 carbon atoms, more typically from 1 to about 12 carbon atoms (C₁-C₁₂ alkyl) with from 1 to about 6 carbon atoms being more preferred. The term “lower alkyl” as used herein includes from 1 to about 6 carbon atoms. Alkyl groups as used herein may optionally include one or more further substituent groups.

As used herein, the term “alkenyl” refers to a straight or branched hydrocarbon chain radical containing up to twenty four carbon atoms and having at least one carbon-carbon double bond. Examples of alkenyl groups include without limitation, ethenyl, propenyl, butenyl, 1-methyl-2-buten-1-yl, dienes such as 1,3-butadiene and the like. Alkenyl groups typically include from 2 to about 24 carbon atoms, more typically from 2 to about 12 carbon atoms with from 2 to about 6 carbon atoms being more preferred. Alkenyl groups as used herein may optionally include one or more further substituent groups.

As used herein, the term “alkynyl” refers to a straight or branched hydrocarbon radical containing up to twenty four carbon atoms and having at least one carbon-carbon triple bond. Examples of alkynyl groups include, without limitation, ethynyl, 1-propynyl, 1-butynyl, and the like. Alkynyl groups typically include from 2 to about 24 carbon atoms, more typically from 2 to about 12 carbon atoms with from 2 to about 6 carbon atoms being more preferred. Alkynyl groups as used herein may optionally include one or more further substituent groups.

As used herein, the term “aliphatic” refers to a straight or branched hydrocarbon radical containing up to twenty four carbon atoms wherein the saturation between any two carbon atoms is a single, double, or triple bond. An aliphatic group preferably contains from 1 to about 24 carbon atoms, more typically from 1 to about 12 carbon atoms with from 1 to about 6 carbon atoms being more preferred. The straight or branched chain of an aliphatic group may be interrupted with one or more heteroatoms that include nitrogen, oxygen, sulfur, and phosphorus. Such aliphatic groups interrupted by heteroatoms include without limitation, polyalkoxys, such as polyalkylene glycols, polyamines, and polyimines. Aliphatic groups as used herein may optionally include further substituent groups.

As used herein, the term “alicyclic” refers to a cyclic ring system wherein the ring is aliphatic. The ring system can comprise one or more rings wherein at least one ring is aliphatic. Preferred alicyclics include rings having from about 5 to about 9 carbon atoms in the ring. Alicyclic as used herein may optionally include further substituent groups.

As used herein, the term “alkoxy”, refers to a radical formed between an alkyl group and an oxygen atom wherein the oxygen atom is used to attach the alkoxy group to a parent molecule. Examples of alkoxy groups include without limitation, methoxy, ethoxy, propoxy, isopropoxy, n-butoxy, sec-butoxy, tert-butoxy, n-pentoxy, neopentoxy, n-hexoxy and the like. Alkoxy groups as used herein may optionally include further substituent groups.

As used herein, the term “aminoalkyl” refers to an amino substituted C₁-C₁₂ alkyl radical. The alkyl portion of the radical forms a covalent bond with a parent molecule. The amino group can be located at any position and the aminoalkyl group can be substituted with a further substituent group at the alkyl and/or amino portions.

As used herein, the terms “aryl” and “aromatic” refer to a mono- or polycyclic carbocyclic ring system radicals having one or more aromatic rings. Examples of aryl groups include without limitation, phenyl, naphthyl, tetrahydronaphthyl, indanyl, idenyl and the like. Preferred aryl ring systems have from about 5 to about 20 carbon atoms in one or more rings. Aryl groups as used herein may optionally include further substituent groups.

As used herein, the terms “aralkyl” and “arylalkyl” refer to an aromatic group that is covalently linked to a C₁-C₁₂ alkyl radical. The alkyl radical portion of the resulting aralkyl (or arylalkyl) group forms a covalent bond with a parent molecule. Examples include without limitation, benzyl, phenethyl and the like. Aralkyl groups as used herein may optionally include further substituent groups attached to the alkyl, the aryl or both groups that form the radical group.

As used herein the term “heterocyclic radical” refers to a radical mono-, or poly-cyclic ring system that includes at least one heteroatom and is unsaturated, partially saturated, or fully saturated, thereby including heteroaryl groups. Heterocyclic is also meant to include fused ring systems wherein one or more of the fused rings contain at least one heteroatom and the other rings can contain one or more heteroatoms or optionally contain no heteroatoms. A heterocyclic radical typically includes at least one atom selected from sulfur, nitrogen, or oxygen. Examples of heterocyclic radicals include, [1,3]dioxolanyl, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, tetrahydrofuryl and the like. Heterocyclic groups as used herein may optionally include further substituent groups.

As used herein the terms “heteroaryl,” and “heteroaromatic,” refer to a radical comprising a mono- or poly-cyclic aromatic ring, ring system or fused ring system wherein at least one of the rings is aromatic and includes one or more heteroatoms. Heteroaryl is also meant to include fused ring systems including systems where one or more of the fused rings contain no heteroatoms. Heteroaryl groups typically include one ring atom selected from sulfur, nitrogen, or oxygen. Examples of heteroaryl groups include without limitation, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl and the like. Heteroaryl radicals can be attached to a parent molecule directly or through a linking moiety such as an aliphatic group or hetero atom. Heteroaryl groups as used herein may optionally include further substituent groups.

As used herein the term “heteroarylalkyl,” refers to a heteroaryl group as previously defined that further includes a covalently attached C₁-C₁₂ alkyl radical. The alkyl radical portion of the resulting heteroarylalkyl group is capable of forming a covalent bond with a parent molecule. Examples include without limitation, pyridinylmethylene, pyrimidinylethylene, napthyridinylpropylene and the like. Heteroarylalkyl groups as used herein may optionally include further substituent groups on one or both of the heteroaryl or alkyl portions.

As used herein the term “acyl,” refers to a radical formed by removal of a hydroxyl group from an organic acid and has the general Formula —C(O)—X where X is typically aliphatic, alicyclic or aromatic. Examples include aliphatic carbonyls, aromatic carbonyls, aliphatic sulfonyls, aromatic sulfinyls, aliphatic sulfinyls, aromatic phosphates, aliphatic phosphates, and the like. Acyl groups as used herein may optionally include further substituent groups.

As used herein the term “hydrocarbyl” includes radical groups that comprise C, O and H. Included are straight, branched, and cyclic groups having any degree of saturation. Such hydrocarbyl groups can include one or more additional heteroatoms selected from N and S and can be further mono or poly substituted with one or more substituent groups.

As used herein the term “mono or poly cyclic structure” is meant to include all ring systems selected from single or polycyclic radical ring systems wherein the rings are fused or linked and is meant to be inclusive of single and mixed ring systems individually selected from aliphatic, alicyclic, aryl, heteroaryl, aralkyl, arylalkyl, heterocyclic, heteroaryl, heteroaromatic and heteroarylalkyl. Such mono and poly cyclic structures can contain rings that each have the same level of saturation or each, independently, have varying degrees of saturation including fully saturated, partially saturated, or fully unsaturated. Each ring can comprise ring atoms selected from C, N, O and S to give rise to heterocyclic rings as well as rings comprising only C ring atoms which can be present in a mixed motif such as for example benzimidazole wherein one ring has only carbon ring atoms and the fused ring has two nitrogen atoms. The mono or poly cyclic structures can be further substituted with substituent groups such as for example phthalimide which has two ═O groups attached to one of the rings. Mono or poly cyclic structures can be attached to parent molecules using various strategies such as directly through a ring atom, fused through multiple ring atoms, through a substituent group or through a bifunctional linking moiety.

As used herein the terms “halo” and “halogen,” refer to an atom selected from fluorine, chlorine, bromine, and iodine.

As used herein the term “oxo” refers to the group (═O).

As used herein the term “protecting group,” refers to a labile chemical moiety which is known in the art to protect reactive groups including without limitation, hydroxyl, amino and thiol groups, against undesired reactions during synthetic procedures. Protecting groups are typically used selectively and/or orthogonally to protect sites during reactions at other reactive sites and can then be removed to leave the unprotected group as is or available for further reactions. Protecting groups as known in the art are described generally in Greene's Protective Groups in Organic Synthesis, 4th edition, John Wiley & Sons, New York, 2007.

Examples of hydroxyl protecting groups include without limitation, acetyl, t-butyl, t-butoxymethyl, methoxymethyl, tetrahydropyranyl, 1-ethoxyethyl, 1-(2-chloroethoxy)ethyl, p-chlorophenyl, 2,4-dinitrophenyl, benzyl, 2,6-dichlorobenzyl, diphenyl methyl, p-nitrobenzyl, bis(2-acetoxyethoxy)methyl (ACE), 2-trimethylsilylethyl, trimethylsilyl, triethylsilyl, t-butyldimethylsilyl, t-butyldiphenylsilyl, triphenylsilyl, [(triisopropylsilyl)oxy]methyl (TOM), benzoylformate, chloroacetyl, trichloroacetyl, trifluoro-acetyl, pivaloyl, benzoyl, p-phenylbenzoyl, 9-fluorenylmethyl carbonate, mesylate, tosylate, triphenylmethyl (trityl), monomethoxytrityl, dimethoxytrityl (DMT), trimethoxytrityl, 1(2-fluorophenyl)-4-methoxypiperidin-4-yl (FPMP), 9-phenylxanthine-9-yl (Pixyl) and 9-(p-methoxyphenyl)xanthine-9-yl (MOX). Wherein more commonly used hydroxyl protecting groups include without limitation, benzyl, 2,6-dichlorobenzyl, t-butyldimethylsilyl, t-butyldiphenylsilyl, benzoyl, mesylate, tosylate, dimethoxytrityl (DMT), 9-phenylxanthine-9-yl (Pixyl) and 9-(p-methoxyphenyl)xanthine-9-yl (MOX).

Examples of amino protecting groups include without limitation, carbamate-protecting groups, such as 2-trimethylsilylethoxycarbonyl (Teoc), 1-methyl-1-(4-biphenylyl)ethoxycarbonyl (Bpoc), t-butoxycarbonyl (BOC), allyloxycarbonyl (Alloc), 9-fluorenylmethyloxycarbonyl (Fmoc), and benzyl-oxycarbonyl (Cbz); amide-protecting groups, such as formyl, acetyl, trihaloacetyl, benzoyl, and nitrophenylacetyl; sulfonamide-protecting groups, such as 2-nitrobenzenesulfonyl; and imine- and cyclic imide-protecting groups, such as phthalimido and dithiasuccinoyl.

Examples of thiol protecting groups include without limitation, triphenylmethyl (trityl), benzyl (Bn), and the like.

As used herein, “administration” or “administering” refers to routes of introducing a compound or composition provided herein to a subject. Examples of routes of administration that can be used include, but are not limited to, administration by inhalation, subcutaneous injection, intrathecal injection, and oral administration.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense compound” means a compound comprising an antisense oligonucleotide and optionally one or more additional features, such as a conjugate group or terminal group.

As used herein, “antisense oligonucleotide” means an oligonucleotide having a nucleobase sequence that is at least partially complementary to a target nucleic acid. Antisense oligonucleotides include but are not limited to antisense RNAi oligonucleotides and antisense RNase H oligonucleotides.

As used herein, “RNAi compound” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics. RNAi compounds may comprise conjugate groups and/or terminal groups. In certain embodiments, an RNAi compound modulates the amount, activity, and/or splicing of a target nucleic acid. The term RNAi compound excludes antisense compounds that act through RNase H.

As used herein, “RNAi oligonucleotide” means an antisense RNAi oligonucleotide or a sense RNAi oligonucleotide.

As used herein, “antisense RNAi oligonucleotide” means an oligonucleotide comprising a “targeting region” that is complementary to a target sequence, and which includes at least one chemical modification suitable for RNAi.

As used herein, “sense RNAi oligonucleotide” means an oligonucleotide comprising an “antisense-hybridizing region” that is complementary to a region of an antisense RNAi oligonucleotide, and which is capable of forming a duplex with such antisense RNAi oligonucleotide. A duplex formed by an antisense RNAi oligonucleotide and a sense RNAi oligonucleotide is referred to as a double-stranded RNAi compound (dsRNAi) or a short interfering RNA (siRNA).

As used herein, “RNase H compound” means an antisense compound that acts, at least in part, through RNase H to modulate a target nucleic acid and/or protein encoded by a target nucleic acid. In certain embodiments, RNase H compounds are single-stranded. In certain embodiments, RNase H compounds are double-stranded. RNase H compounds may comprise conjugate groups and/or terminal groups. In certain embodiments, an RNase H compound modulates the amount or activity of a target nucleic acid. The term RNase H compound excludes antisense compounds that act principally through RISC/Ago2.

As used herein, “antisense RNase H oligonucleotide” means an oligonucleotide comprising a region that is complementary to a target sequence, and which includes at least one chemical modification suitable for RNase H-mediated nucleic acid reduction.

As used herein, “bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety, and the bicyclic sugar moiety is a modified bicyclic furanosyl sugar moiety. In certain embodiments, the bicyclic sugar moiety does not comprise a furanosyl moiety.

As used herein, “cEt” or “constrained ethyl” means a bicyclic sugar moiety, wherein the first ring of the bicyclic sugar moiety is a ribosyl sugar moiety, the second ring of the bicyclic sugar is formed via a bridge connecting the 4′-carbon and the 2′-carbon, the bridge has the formula 4′-CH(CH₃)—O-2′, and the methyl group of the bridge is in the S configuration. A cEt bicyclic sugar moiety is in the β-D configuration.

As used herein, “complementary” in reference to an oligonucleotide means that at least 70% of the nucleobases of such oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases are nucleobase pairs that are capable of forming hydrogen bonds with one another. Complementary nucleobase pairs include adenine (A) and thymine (T), adenine (A) and uracil (U), cytosine (C) and guanine (G), 5-methyl cytosine (^{m}C) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that such oligonucleotides are complementary to another oligonucleotide or nucleic acid at each nucleoside of the oligonucleotide.

As used herein, “conjugate group” means a group of atoms that is directly or indirectly attached to an oligonucleotide. Conjugate groups may comprise a conjugate moiety and a conjugate linker that attaches the conjugate moiety to the oligonucleotide.

As used herein, “conjugate linker” means a bond or a group of atoms comprising at least one bond that connects a conjugate moiety to an oligonucleotide.

As used herein, “conjugate moiety” means a group of atoms that is attached to an oligonucleotide via a conjugate linker.

As used herein, “cytotoxic” or “cytotoxicity” in the context of an effect of an oligomeric compound or a parent oligomeric compound on cultured cells means an at least 2-fold increase in caspase activation following administration of 10 μM or less of the oligomeric compound or parent oligomeric compound to the cultured cells relative to cells cultured under the same conditions but that are not administered the oligomeric compound or parent oligomeric compound. In certain embodiments, cytotoxicity is measured using a standard in vitro cytotoxicity assay.

As used herein, “deoxy region” means a region of 5-12 contiguous nucleotides, wherein at least 70% of the nucleosides are stereo-standard DNA nucleosides. In certain embodiments, each nucleoside is selected from a stereo-standard DNA nucleoside (a nucleoside comprising a β-D-2′-deoxyribosyl sugar moiety), a stereo-non-standard nucleoside of Formula I-VII, a bicyclic nucleoside, and a substituted stereo-standard nucleoside. In certain embodiments, a deoxy region supports RNase H activity. In certain embodiments, a deoxy region is the gap of a gapmer.

As used herein, “double-stranded antisense compound” means an antisense compound comprising two oligomeric compounds that are complementary to each other and form a duplex, and wherein one of the two said oligomeric compounds comprises an antisense oligonucleotide.

As used herein, “expression” includes all the functions by which a gene's coded information is converted into structures present and operating in a cell. Such structures include, but are not limited to, the products of transcription and translation. As used herein, “modulation of expression” means any change in amount or activity of a product of transcription or translation of a gene. Such a change may be an increase or a reduction of any amount relative to the expression level prior to the modulation.

As used herein, “gapmer” means an oligonucleotide having a central region comprising a plurality of nucleosides that support RNase H cleavage positioned between a 5′-region and a 3′-region. In certain embodiments, the nucleosides of the 5′-region and 3′-region each comprise a 2′-substituted furanosyl sugar moiety or a bicyclic sugar moiety, and the 3′- and 5′-most nucleosides of the central region each comprise a sugar moiety independently selected from a 2′-deoxyfuranosyl sugar moiety or a sugar surrogate. The positions of the central region refer to the order of the nucleosides of the central region and are counted starting from the 5′-end of the central region. Thus, the 5′-most nucleoside of the central region is at position 1 of the central region. The “central region” may be referred to as a “gap”, and the “5′-region” and “3′-region” may be referred to as “wings”. Gaps of gapmers are deoxy regions.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “inhibiting the expression or activity” refers to a reduction or blockade of the expression or activity relative to the expression or activity in an untreated or control sample and does not necessarily indicate a total elimination of expression or activity.

As used herein, the terms “internucleoside linkage” means a group of atoms or bond that forms a covalent linkage between adjacent nucleosides in an oligonucleotide. As used herein “modified internucleoside linkage” means any internucleoside linkage other than a naturally occurring, phosphodiester internucleoside linkage. “Phosphorothioate linkage” means a modified internucleoside linkage in which one of the non-bridging oxygen atoms of a phosphodiester is replaced with a sulfur atom. Modified internucleoside linkages may or may not contain a phosphorus atom. A “neutral internucleoside linkage” is a modified internucleoside linkage that does not have a negatively charged phosphate in a buffered aqueous solution at pH=7.0. A “backbone constrained internucleoside linkage” is an internucleoside linkage that comprises a carbon or heterocycle chain that forms a covalent linkage with an adjacent internucleoside linkage, which is also a “backbone constrained internucleoside linkage”.

As used herein, “linked nucleosides” are nucleosides that are connected in a continuous sequence (i.e. no additional nucleosides are present between those that are linked).

As used herein, “maximum tolerated dose” means the highest dose of a compound that does not cause unacceptable side effects. In certain embodiments, the maximum tolerated dose is the highest dose of a modified oligonucleotide that does not cause an ALT elevation of three times the upper limit of normal as measured by a standard assay.

As used herein, “mismatch” or “non-complementary” means a nucleobase of a first oligonucleotide that is not complementary with the corresponding nucleobase of a second oligonucleotide or target nucleic acid when the first and second oligomeric compound are aligned.

As used herein, “modulating” refers to changing or adjusting a feature in a cell, tissue, organ, or organism.

As used herein, “MOE” means methoxyethyl. “2′-MOE” or “2′-O-methoxyethyl” means a 2′-OCH₂CH₂OCH₃ group at the 2′-position of a furanosyl ring. In certain embodiments, the 2′-OCH₂CH₂OCH₃ group is in place of the 2′-OH group of a ribosyl ring or in place of a 2′-H in a 2′-deoxyribosyl ring.

As used herein, “motif” means the pattern of unmodified and/or modified sugar moieties, nucleobases, and/or internucleoside linkages, in an oligonucleotide.

As used herein, “naturally occurring” means found in nature.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a modified nucleobase is a group of atoms capable of pairing with at least one unmodified nucleobase. A universal base is a nucleobase that can pair with any one of the five unmodified nucleobases. 5-methylcytosine (^{m}C) is one example of a modified nucleobase.

As used herein, “nucleobase sequence” means the order of contiguous nucleobases in a nucleic acid or oligonucleotide independent of any sugar moiety or internucleoside linkage modification.

As used herein, “nucleoside” means a moiety comprising a nucleobase and a sugar moiety. The nucleobase and sugar moiety are each, independently, unmodified or modified. As used herein, “modified nucleoside” means a nucleoside comprising a modified nucleobase and/or a modified sugar moiety.

As used herein, “oligomeric compound” means a compound consisting of (1) an oligonucleotide (a single-stranded oligomeric compound) or two oligonucleotides hybridized to one another (a double-stranded oligomeric compound); and (2) optionally one or more additional features, such as a conjugate group or terminal group which may be bound to the oligonucleotide of a single-stranded oligomeric compound or to one or both oligonucleotides of a double-stranded oligomeric compound.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 12-30 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, liquids, powders, or suspensions that can be aerosolized or otherwise dispersed for inhalation by a subject. In certain embodiments, a pharmaceutically acceptable carrier or diluent is sterile water; sterile saline; or sterile buffer solution.

As used herein “pharmaceutically acceptable salts” means physiologically and pharmaceutically acceptable salts of compounds, such as oligomeric compounds, i.e., salts that retain the desired biological activity of the compound and do not impart undesired toxicological effects thereto.

As used herein “pharmaceutical composition” means a mixture of substances suitable for administering to a subject. For example, a pharmaceutical composition may comprise an antisense compound and an aqueous solution.

As used herein, the term “single-stranded” in reference to an antisense compound means such a compound consists of one oligomeric compound that is not paired with a second oligomeric compound to form a duplex. “Self-complementary” in reference to an oligonucleotide means an oligonucleotide that at least partially hybridizes to itself. A compound consisting of one oligomeric compound, wherein the oligonucleotide of the oligomeric compound is self-complementary, is a single-stranded compound. A single-stranded antisense or oligomeric compound may be capable of binding to a complementary oligomeric compound to form a duplex, in which case the compound would no longer be single-stranded.

As used herein, “subject” means a human or non-human animal selected for treatment or therapy.

As used herein, “sugar moiety” means an unmodified sugar moiety or a modified sugar moiety. As used herein, “unmodified sugar moiety” means a β-D-ribosyl moiety, as found in naturally occurring RNA, or a β-D-2′-deoxyribosyl sugar moiety as found in naturally occurring DNA. As used herein, “modified sugar moiety” or “modified sugar” means a sugar surrogate or a furanosyl sugar moiety other than a β-D-ribosyl or a β-D-2′-deoxyribosyl. Modified furanosyl sugar moieties may be modified or substituted at a certain position(s) of the sugar moiety, or unsubstituted, and they may or may be stereo-non-standard sugar moieties. Modified furanosyl sugar moieties include bicyclic sugars and non-bicyclic sugars. As used herein, “sugar surrogate” means a modified sugar moiety that does not comprise a furanosyl or tetrahydrofuranyl ring (is not a “furanosyl sugar moiety”) and that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “target nucleic acid,” “target RNA,” “target RNA transcript” and “nucleic acid target” means a nucleic acid that an oligomeric compound, such as an antisense compound, is designed to affect. In certain embodiments, an oligomeric compound comprises an oligonucleotide having a nucleobase sequence that is complementary to more than one RNA, only one of which is the target RNA of the oligomeric compound. In certain embodiments, the target RNA is an RNA present in the species to which an oligomeric compound is administered.

As used herein, “therapeutic index” means a comparison of the amount of a compound that causes a therapeutic effect to the amount that causes toxicity. Compounds having a high therapeutic index have strong efficacy and low toxicity. In certain embodiments, increasing the therapeutic index of a compound increases the amount of the compound that can be safely administered.

As used herein, “treat” refers to administering a compound or pharmaceutical composition to an animal in order to affect an alteration or improvement of a disease, disorder, or condition in the animal.

### CERTAIN EMBODIMENTS

The present disclosure provides the following non-limiting numbered embodiments:
Embodiment 1. A compound comprising the formula:

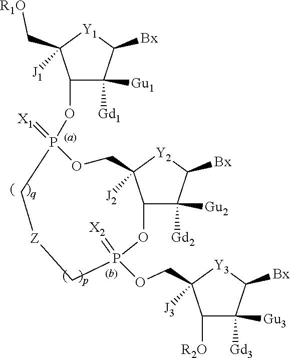

Formula I
wherein each Bx is, independently, a heterocyclic base moiety;
R₁ is H, a hydroxyl protecting group or a conjugate group;
R₂ is H, a hydroxyl protecting group, a conjugate group, or a reactive phosphorous group;
(a) and (b) are the stereochemistry at the phosphate and are independently selected from (R), (S), and (R,S);
p is from 1 to 7;
q is from 1 to 7;
and wherein p+q is greater than or equal to 4;
Z is CH₂ or O;
each X is, independently, O or S;
each Y is, independently, O or S;
either J₁ and G_{d1} form a J₁ and G_{d1} bridge and Gᵤ₁ is H, or J₁ is H and G_{d1} and Gᵤ₁ are independently selected from H, OH, halogen or O—[C(R₃)(R₄)]ₙ—[(C═O)ₘ—X^{G}]ⱼ—R₅;
either J₂ and G_{d2} form a J₂ and G_{d2} bridge and Gᵤ₁ is H, or J₂ is H and G_{d2} and Gᵤ₂ are independently selected from H, OH, halogen or O—[C(R₃)(R₄)]ₙ—[(C═O)ₘ—X^{G}]ⱼ—R₅;
either J₃ and G_{d3} form a J₃ and G_{d3} bridge and Gᵤ₁ is H, or J₃ is H and G_{d3} and Gᵤ₃ are independently selected from H, OH, halogen or O—[C(R₃)(R₄)]ₙ—[(C═O)ₘ—X^{G}]ⱼ—R₅;
   wherein each J to G_{d} bridge has a formula independently selected from —CH(CH₃)—O— or —(CH₂)ₖ—O—, wherein k is from 1 to 3;
each R₃ and R₄ is, independently, H, halogen, C₁-C₆ alkyl or substituted C₁-C₆ alkyl;
   each X^{G} is O, S or N(E₁);
   R₅ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
   E₁, E₂ and E₃ are each, independently, H, C₁-C₆ alkyl or substituted C₁-C₆ alkyl;
   n is from 1 to 6;
   m is 0 or 1;
   j is 0 or 1;
   each substituted group comprises one or more optionally protected substituent groups independently selected from halogen, OJ₁, N(J₁)(J₂), =NJ₁, SJ₁, N₃, CN, OC(═X₂)J₁, OC(═X₂)N(J₁)(J₂) and C(=Q₂)N(J₁)(J₂);
   Q₂ is O, S or NJ₃;
   each J₁, J₂ and J₃ is, independently, H or C₁-C₆ alkyl.
Embodiment 2. The compound of claim 1, wherein each Y is O.
Embodiment 3. The compound of claim 1 or 2, wherein each X is S.
Embodiment 4. The compound of claim 1 or 2, wherein each X is O.
Embodiment 5. The compound of any of claims 1-4, wherein p+q equals 5.
Embodiment 6. The compound of any of claims 1-4, wherein p+q equals 6.
Embodiment 7. The compound of any of claims 1-4, wherein p+q equals 7.
Embodiment 8. The compound of any of claims 1-4, wherein p+q equals 8.
Embodiment 9. The compound of any of claims 1-8, wherein R₁ and R₂ are independently, hydrogen or a hydroxyl protecting group.
Embodiment 10. The compound of any of claims 1-8, wherein R₁ is a hydroxyl protecting group and R₂ is a reactive phosphorous group.
Embodiment 11. The compound of any of claims 1-10, wherein each Gᵤ is H.
Embodiment 12. The compound of any of claims 1-11, wherein each G_{d} is independently selected from H, O-methoxyethyl, O-methyl, or fluoro.
Embodiment 13. The compound of any of claims 1-11, wherein each J forms a bridge with each G_{d}.
Embodiment 14. The compound of claim 13, wherein the bridge has the formula —(CH₂)—O—.
Embodiment 15. The compound of any of claims 1-14, wherein each Bx is, independently, an optionally protected pyrimidine, substituted pyrimidine, purine or substituted purine.
Embodiment 16. The compound of any of claims 1-15, wherein each Bx is, independently, a uracil, thymine, cytosine, 5-methyl-cytosine, adenine, or guanine.
Embodiment 17. The compound of any of claims 1-16, wherein (a) is (R).
Embodiment 18. The compound of any of claims 1-16, wherein (a) is (S).
Embodiment 19. The compound of any of claims 1-18, wherein (b) is (R).
Embodiment 20. The compound of any of claims 1-18, wherein (b) is (S).
Embodiment 21. The compound of any of claims 1-16, wherein (a) is (R) and (b) is (R).
Embodiment 22. An oligomeric compound comprising a modified oligonucleotide consisting of 8 to 40 linked nucleosides, wherein the modified oligonucleotide comprises a region having the formula:

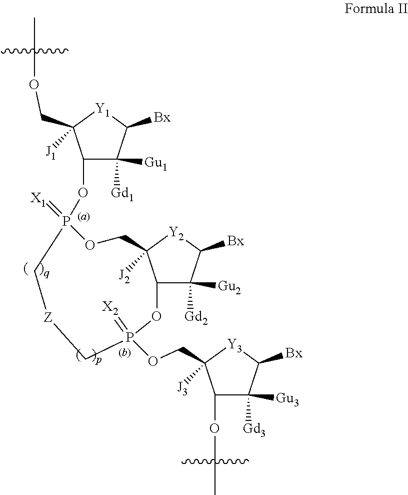

wherein each Bx is, independently, a heterocyclic base moiety;
(a) and (b) are the stereochemistry at the phosphate and are independently selected from (R), (S), and (R,S);
p is from 1 to 7;
q is from 1 to 7;
and wherein p+q is greater than or equal to 4;
Z is CH₂ or O;
each X is, independently, O or S;
each Y is, independently, O or S;
either J₁ and G_{d1} form a J₁ and G_{d1} bridge and Gᵤ₁ is H, or J₁ is H and G_{d1} and Gᵤ₁ are independently selected from H, OH, halogen or O—[C(R₃)(R₄)]ₙ—[(C═O)ₘ—X^{G}]ⱼ—R₅;
either J₂ and G_{d2} form a J₂ and G_{d2} bridge and Gᵤ₁ is H, or J₂ is H and G_{d2} and Gᵤ₂ are independently selected from H, OH, halogen or O—[C(R₃)(R₄)]ₙ—[(C═O)ₘ—X^{G}]ⱼ—R₅;
either J₃ and G_{d3} form a J₃ and G_{d3} bridge and Gᵤ₁ is H, or J₃ is H and G_{d3} and Gᵤ₃ are independently selected from H, OH, halogen or O—[C(R₃)(R₄)]ₙ—[(C═O)ₘ—X^{G}]ⱼ—R₅;
   wherein each J to G_{d} bridge has a formula independently selected from —CH(CH₃)—O— or —(CH₂)ₖ—O—, wherein k is from 1 to 3;
each R₃ and R₄ is, independently, H, halogen, C₁-C₆ alkyl or substituted C₁-C₆ alkyl;
   each X^{G} is O, S or N(E₁);
   R₅ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
   E₁, E₂ and E₃ are each, independently, H, C₁-C₆ alkyl or substituted C₁-C₆ alkyl;
   n is from 1 to 6;
   m is 0 or 1;
   j is 0 or 1;
   each substituted group comprises one or more optionally protected substituent groups independently selected from halogen, OJ₁, N(J₁)(J₂), =NJ₁, SJ₁, N₃, CN, OC(═X₂)J₁, OC(═X₂)N(J₁)(J₂) and C(=Q₂)N(J₁)(J₂);
   Q₂ is O, S or NJ₃;
   each J₁, J₂ and J₃ is, independently, H or C₁-C₆ alkyl.
Embodiment 23. The oligomeric compound of claim 22, wherein each Y is O.
Embodiment 24. The oligomeric compound of claim 22 or 23, wherein each X is S.
Embodiment 25. The oligomeric compound of claim 22 or 23, wherein each X is O.
Embodiment 26. The oligomeric compound of any of claims 22-25, wherein p+q equals 5.
Embodiment 27. The oligomeric compound of any of claims 22-26, wherein p+q equals 6.
Embodiment 28. The oligomeric compound of any of claims 22-27, wherein p+q equals 7.
Embodiment 29. The oligomeric compound of any of claims 22-28, wherein p+q equals 8.
Embodiment 30. The oligomeric compound of any of claims 22-29, wherein each Gᵤ is H.
Embodiment 31. The oligomeric compound of any of claims 22-30, wherein each G_{d} is independently selected from H, O-methoxyethyl, O-methyl, or fluoro.
Embodiment 32. The oligomeric compound of any of claims 22-30, wherein each J forms a bridge with each G_{d}.
Embodiment 33. The oligomeric compound of claim 32, wherein the bridge has the formula —(CH₂)—O—.
Embodiment 34. The oligomeric compound of any of claims 22-33, wherein each Bx is, independently, an optionally protected pyrimidine, substituted pyrimidine, purine or substituted purine.
Embodiment 35. The oligomeric compound of any of claims 22-34, wherein each Bx is, independently, a uracil, thymine, cytosine, 5-methyl-cytosine, adenine, or guanine.
Embodiment 36. The oligomeric compound of any of claims 22-35, wherein (a) is (R).
Embodiment 37. The oligomeric compound of any of claims 22-35, wherein (a) is (S).
Embodiment 38. The oligomeric compound of any of claims 22-37, wherein (b) is (R).
Embodiment 39. The oligomeric compound of any of claims 22-37, wherein (b) is (S).
Embodiment 40. The oligomeric compound of any of claims 22-35, wherein (a) is (S) and (b) is (R).
Embodiment 41. The oligomeric compound of any of claims 22-40, wherein the modified oligonucleotide consists of 12-24, 12-20, 16-20, 18-20, or 22-23 linked nucleosides.
Embodiment 42. The oligomeric compound of any of claims 22-40, wherein the modified oligonucleotide consists of 16 linked nucleosides.
Embodiment 43. The oligomeric compound of any of claims 22-40, wherein the modified oligonucleotide consists of 18 linked nucleosides.
Embodiment 44. The oligomeric compound of any of claims 22-40, wherein the modified oligonucleotide consists of 20 linked nucleosides.
Embodiment 45. The oligomeric compound of any of claims 22-40, wherein the modified oligonucleotide consists of 23 linked nucleosides.
Embodiment 46. The oligomeric compound of any of claims 22-45, wherein at least one internucleoside linkage of the modified oligonucleotide that is not part of the region having Formula II is a modified internucleoside linkage.
Embodiment 47. The oligomeric compound of any of claims 22-45, wherein each internucleoside linkage of the modified oligonucleotide that is not part of the region having Formula II is a modified internucleoside linkage.
Embodiment 48. The oligomeric compound of claim 46 or 47, wherein the modified internucleoside linkage is a phosphorothioate internucleoside linkage.
Embodiment 49. The oligomeric compound of any of claims 22-45, wherein each internucleoside linkage or the modified oligonucleotide that is not a part of the region having Formula II is independently selected from a phosphodiester internucleoside linkage and a phosphorothioate internucleoside linkage.
Embodiment 50. The oligomeric compound of claim 46, wherein each internucleoside linkage of the modified oligonucleotide that is not part of the region having Formula II is independently selected from a phosphodiester internucleoside linkage and a phosphorothioate internucleoside linkage.
Embodiment 51. The oligomeric compound of any of claims 22-50, wherein the region of the modified oligonucleotide having Formula II is at the 5′-end of the modified oligonucleotide.
Embodiment 52. The oligomeric compound of any of claims 22-50, wherein the region of the modified oligonucleotide having Formula II is at the 3′-end of the modified oligonucleotide.
Embodiment 53. The oligomeric compound of any of claims 22-50, wherein the region of the modified oligonucleotide having Formula II is not at a terminus of the modified oligonucleotide.
Embodiment 54. The oligomeric compound of any of claims 22-53, wherein the modified oligonucleotide has a sugar motif comprising:
   a 5′-region consisting of 1-6 linked 5′-region nucleosides;
   a central region consisting of 6-10 linked central region nucleosides; and
   a 3′-region consisting of 1-6 linked 3′-region nucleosides; wherein
   each of the 5′-region nucleosides and each of the 3′-region nucleosides comprises a modified sugar moiety and each of the central region nucleosides comprises a 2′-β-D-deoxyribosyl sugar moiety.
Embodiment 55. The oligomeric compound of claim 54, wherein the modified oligonucleotide has a sugar motif comprising:
   a 5′-region consisting of 5 linked 5′-region nucleosides;
   a central region consisting of 10 linked central region nucleosides; and
   a 3′-region consisting of 5 linked 3′-region nucleosides; wherein
   each of the 5′-region nucleosides and each of the 3′-region nucleosides comprises a 2′-MOE modified sugar moiety and each of the central region nucleosides comprises a 2′-β-D-deoxyribosyl sugar moiety.
Embodiment 56. The oligomeric compound of any of claims 22-53, wherein the oligomeric compound comprises an antisense RNAi oligonucleotide comprising a targeting region comprising at least 15 contiguous nucleobases, wherein the targeting region is at least 90% complementary to an equal-length portion of a target RNA.
Embodiment 57. The oligomeric compound of any of claims 22-53, wherein the oligomeric compound comprises a sense RNAi oligonucleotide.
Embodiment 58. The oligomeric compound of any of claims 22-57, comprising a conjugate group comprising a conjugate moiety and a conjugate linker.
Embodiment 59. The oligomeric compound of any of claims 22-58, comprising a terminal group.
Embodiment 60. The oligomeric compound of claim 60, wherein the terminal group is a 5′ stabilized phosphate.
Embodiment 61. An oligomeric duplex, comprising a first oligomeric compound comprising an antisense RNAi oligonucleotide of claim 56 and a second oligomeric compound comprising a sense RNAi oligonucleotide consisting of 17 to 30 linked nucleosides, wherein the nucleobase sequence of the sense RNAi oligonucleotide comprises an antisense-hybridizing region having at least 15 contiguous nucleobases wherein the antisense-hybridizing region is at least 90% complementary to an equal length portion of the antisense RNAi oligonucleotide.
Embodiment 62. An oligomeric duplex, comprising a first oligomeric compound comprising an antisense RNAi oligonucleotide comprising a targeting region having at least 15 contiguous nucleobases, wherein the targeting region is at least 90% complementary to an equal-length portion of a target RNA; and a second oligomeric compound comprising a sense RNAi oligonucleotide of claim 57, wherein the nucleobase sequence of the sense RNAi oligonucleotide comprises an antisense-hybridizing region comprising least 15 contiguous nucleobases wherein the antisense-hybridizing region is at least 90% complementary to an equal length portion of the antisense RNAi oligonucleotide.
Embodiment 63. The oligomeric duplex of claim 61 or 62, wherein the first oligomeric compound and/or the second oligomeric compound comprises a conjugate group comprising a conjugate moiety and a conjugate linker.

### I. Certain Oligonucleotides

In certain embodiments, provided herein are oligomeric compounds comprising modified oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage.

A. Certain Modified Nucleosides

Modified nucleosides comprise a modified sugar moiety or a modified nucleobase or both a modified sugar moiety and a modified nucleobase.

1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more substituent groups none of which bridges two atoms of the furanosyl ring to form a bicyclic structure. Such non bridging substituents may be at any position of the furanosyl, including but not limited to substituents at the 2′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE” or “O-methoxyethyl”), and 2′-O—N-alkyl acetamide, e.g., 2′-O—N-methyl acetamide (“NMA”), 2′-O—N-dimethyl acetamide, 2′-O—N-ethyl acetamide, or 2′-O—N-propyl acetamide. For example, see U.S. Pat. No. 6,147,200, Prakash et al., 2003, Org. Lett., 5, 403-6. A “2′-O—N-methyl acetamide nucleoside” or “2′-NMA nucleoside” is shown below:

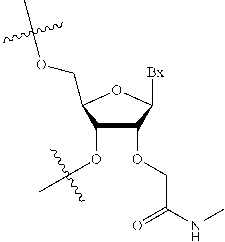

In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂), ON(CH₃)₂, O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl, e.g., for example, OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂₀N(CH₃)₂, O(CH₂)₂O(CH₂)₂N(CH₃)₂, and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, OCH₂CH₂OCH₃, and OCH₂C(═O)—N(H)CH₃.

Certain modified sugar moieties comprise a substituent that bridges two atoms of the furanosyl ring to form a second ring, resulting in a bicyclic sugar moiety. In certain such embodiments, the bicyclic sugar moiety comprises abridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt”), 4′-CH₂—O—CH₂-2′, 4′-CH₂—NI-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C—(H)(CH₃)-2′ (see, e.g., Zhou, et al., J Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—NI-2′, and 4′-CH₂—NI—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J Org. Chem., 1998, 63, 10035-10039; Srivastava et al., J Am. Chem. Soc., 2007, 129, 8362-8379; Wengel et al., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

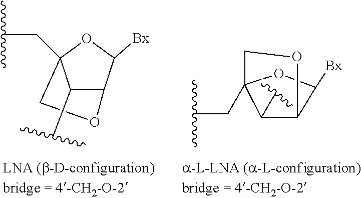

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon, or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

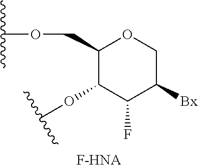

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

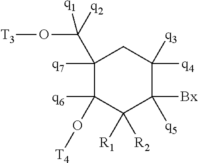

wherein, independently, for each of said modified THP nucleoside:

Bx is a nucleobase moiety;

T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group;

q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and

each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

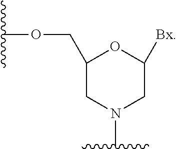

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

2. Certain Modified Nucleobases

In certain embodiments, modified oligonucleotides comprise one or more nucleosides comprising an unmodified nucleobase. In certain embodiments, modified oligonucleotides comprise one or more nucleosides comprising a modified nucleobase. In certain embodiments, modified oligonucleotides comprise one or more nucleosides that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyl adenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., U.S. Pat. No. 6,166,199; and Matteucci et al., U.S. Pat. No. 6,005,096.

B. Certain Modified Internucleoside Linkages

In certain embodiments, compounds described herein having one or more modified internucleoside linkages are selected over compounds having only phosphodiester internucleoside linkages because of desirable properties such as, for example, enhanced cellular uptake, enhanced affinity for target nucleic acids, and increased stability in the presence of nucleases.

In certain embodiments, compounds comprise or consist of a modified oligonucleotide complementary to a target nucleic acid comprising one or more modified internucleoside linkages. In certain embodiments, the modified internucleoside linkages are phosphorothioate linkages.

In certain embodiments, compounds comprise two backbone constrained internucleoside linkages that form a macrocycle with each other to generate a compound comprising the formula below:

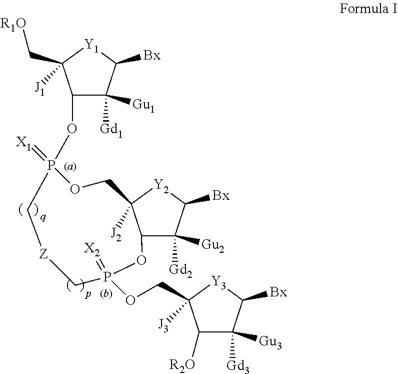

wherein each Bx is, independently, a heterocyclic base moiety;
R₁ is H, a hydroxyl protecting group or a conjugate group;
R₂ is H, a hydroxyl protecting group, a conjugate group, or a reactive phosphorous group;
(a) and (b) are the stereochemistry at the phosphate and are independently selected from (R), (S), and (R,S);
p is from 1 to 7;
q is from 1 to 7;
and wherein p+q is greater than or equal to 4;
Z is CH₂ or 0;
each X is, independently, O or S;
each Y is, independently, O or S;
either J₁ and G_{d1} form a J₁ and G_{d1} bridge and Gᵤ₁ is H, or J₁ is H and G_{d1} and Gᵤ₁ are independently selected from H, OH, halogen or O—[C(R₃)(R₄)]ₙ—[(C═O)ₘ—X^{G}]ⱼ—R₅;
either J₂ and G_{d2} form a J₂ and G_{d2} bridge and Gᵤ₁ is H, or J₂ is H and G_{d2} and Gᵤ₂ are independently selected from H, OH, halogen or O—[C(R₃)(R₄)]ₙ—[(C═O)ₘ—X^{G}]ⱼ—R₅;
either J₃ and G_{d3} form a J₃ and G_{d3} bridge and Gᵤ₁ is H, or J₃ is H and G_{d3} and Gᵤ₃ are independently selected from H, OH, halogen or O—[C(R₃)(R₄)]ₙ—[(C═O)ₘ—X^{G}]ⱼ—R₅;
wherein each J to G_{d} bridge has a formula independently selected from —CH(CH₃)—O— or —(CH₂)ₖ—O—,
   wherein k is from 1 to 3;
   each R₃ and R₄ is, independently, H, halogen, C₁-C₆ alkyl or substituted C₁-C₆ alkyl;
each X^{G} is O, S or N(E₁);
R₅ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(═X₂)J₁, OC(═X₂)N(J₁)(J₂) and C(=Q₂)N(J₁)(J₂);
Q₂ is O, S or NJ₃;
each J₁, J₂ and J₃ is, independently, H or C₁-C₆ alkyl.

In certain embodiments, the present disclosure provides a compound comprising a modified oligonucleotide comprising 8 to 40 linked nucleosides, wherein the modified oligonucleotide comprises at least two backbone constrained internucleoside linkages that form a macrocycle with each other to generate an oligonucleotide that comprises at least one region having the formula:

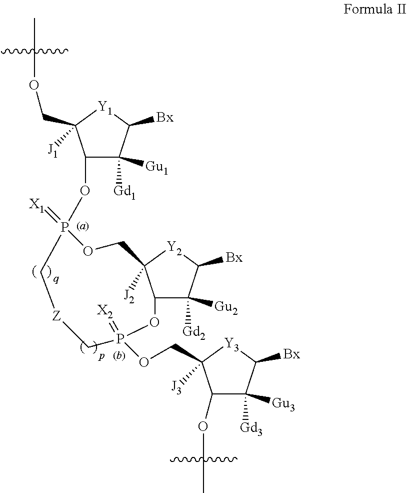

wherein each Bx is, independently, a heterocyclic base moiety;
(a) and (b) are the stereochemistry at the phosphate and are independently selected from (R), (S), and (R,S);
p is from 1 to 7;
q is from 1 to 7;
and wherein p+q is greater than or equal to 4;
Z is CH₂ or 0;
each X is, independently, O or S;
each Y is, independently, O or S;
either J₁ and G_{d1} form a J₁ and G_{d1} bridge and Gᵤ₁ is H, or J₁ is H and G_{d1} and Gᵤ₁ are independently selected from H, OH, halogen or O—[C(R₃)(R₄)]ₙ—[(C═O)ₘ—X^{G}]ⱼ—R₅;
either J₂ and G_{d2} form a J₂ and G_{d2} bridge and Gᵤ₁ is H, or J₂ is H and G_{d2} and Gᵤ₂ are independently selected from H, OH, halogen or O—[C(R₃)(R₄)]ₙ—[(C═O)ₘ—X^{G}]ⱼ—R₅;
either J₃ and G_{d3} form a J₃ and G_{d} bridge and Gᵤ₁ is H, or J₃ is H and G_{d3} and Gᵤ₃ are independently selected from H, OH, halogen or O—[C(R₃)(R₄)]ₙ—[(C═O)ₘ—X^{G}]ⱼ—R₅;
wherein each J to G_{d} bridge has a formula independently selected from —CH(CH₃)—O— or —(CH₂)ₖ—O—,
   wherein k is from 1 to 3;
   each R₃ and R₄ is, independently, H, halogen, C₁-C₆ alkyl or substituted C₁-C₆ alkyl;
each X^{G} is O, S or N(E₁);
R₅ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(═X₂)J₁, OC(═X₂)N(J₁)(J₂) and C(=Q₂)N(J₁)(J₂);
Q₂ is O, S or NJ₃;
each J₁, J₂ and J₃ is, independently, H or C₁-C₆ alkyl.

In certain embodiments, nucleosides of modified oligonucleotides may be linked together using any internucleoside linkage. The two main classes of internucleoside linkages are defined by the presence or absence of a phosphorus atom. Representative phosphorus-containing internucleoside linkages include unmodified phosphodiester internucleoside linkages, modified phosphotriesters such as THP phosphotriester and isopropyl phosphotriester, phosphonates such as methylphosphonate, isopropyl phosphonate, isobutyl phosphonate, and phosphonoacetate, phosphoramidates, phosphorothioate, and phosphorodithioate (“HS-P=S”). Representative non-phosphorus containing internucleoside linkages include but are not limited to methylenemethylimino (—CH₂—N(CH₃)—O—CH₂—), thiodiester, thionocarbamate (—O—C(═O)(NH)—S—); siloxane (—O—SiH₂—O—); formacetal, thioacetamido (TANA), alt-thioformacetal, glycine amide, and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. All phosphorothioate linkages described herein are stereorandom unless otherwise specified. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

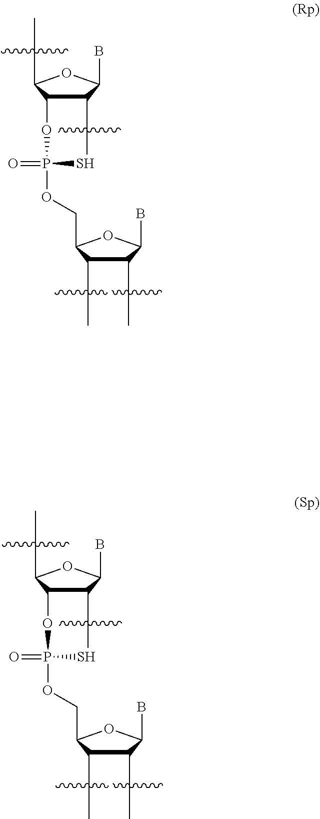

Unless otherwise indicated, chiral internucleoside linkages of modified oligonucleotides described herein can be stereorandom or in a particular stereochemical configuration.

Neutral internucleoside linkages include, without limitation, phosphotriesters, phosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′- O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, nucleic acids can be linked 2′ to 5′ rather than the standard 3′ to 5′ linkage. Such a linkage is illustrated below.

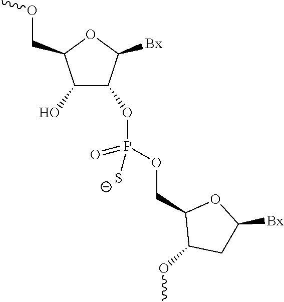

In certain embodiments, nucleosides can be linked by vicinal 2′, 3′-phosphodiester bonds. In certain such embodiments, the nucleosides are threofuranosyl nucleosides (TNA; see Bala, et al., J Org. Chem. 2017, 82:5910-5916). A TNA linkage is shown below.

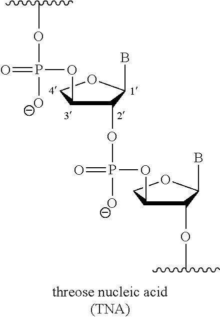

Additional modified linkages include a, β-D-CNA type linkages and related conformationally-constrained linkages, shown below. Synthesis of such molecules has been described previously (see Dupouy, et al., Angew. Chem Int. Ed. Engl. 2014, 45: 3623-3627; Borsting, et al. Tetahedron, 2004, 60:10955-10966; Ostergaard, et al., ACS Chem. Biol. 2014, 9: 1975-1979; Dupouy, et al., Eur. J. Org. Chem., 2008, 1285-1294; Martinez, et al., PLoS One, 2011, 6:e25510; Dupouy, et al., Eur. J. Org. Chem., 2007, 5256˜5264; Boissonnet, et al., New J. Chem., 2011 35: 1528-1533.)

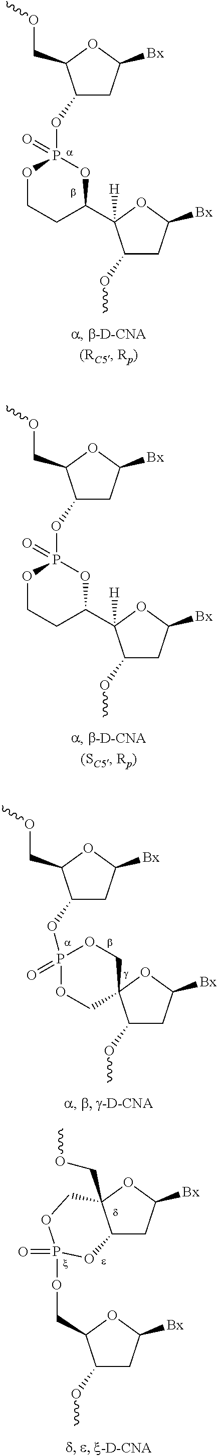

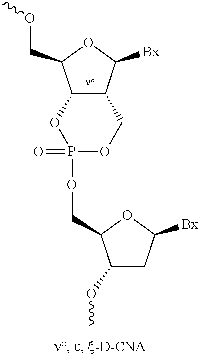

### II. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

A. Certain Motifs

1. Certain Sugar Motifs

In certain embodiments, oligonucleotides comprise one or more type of modified sugar and/or unmodified sugar moiety arranged along the oligonucleotide, or portion thereof, in a defined pattern or sugar motif. In certain instances, such sugar motifs include but are not limited to any of the sugar modifications discussed herein.

In certain embodiments, modified oligonucleotides have a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-6 nucleosides. In certain embodiments, each nucleoside of each wing of a gapmer comprises a modified sugar moiety. In certain embodiments, at least one, at least two, at least three, at least four, at least five, or at least six nucleosides of each wing of a gapmer comprises a modified sugar moiety.

In certain embodiments, the gap of a gapmer comprises 7-12 nucleosides. In certain embodiments, each nucleoside of the gap of a gapmer comprises a 2′-deoxyribosyl sugar moiety. In certain embodiments, at least one nucleoside of the gap of a gapmer comprises a modified sugar moiety and each remaining nucleoside comprises a 2′-deoxyribosyl sugar moiety.

Herein, the lengths (number of nucleosides) of the three regions of a gapmer may be provided using the notation [# of nucleosides in the 5′-wing]-[# of nucleosides in the gap]-[# of nucleosides in the 3′-wing]. Thus, a 5-10-5 gapmer consists of 5 linked nucleosides in each wing and 10 linked nucleosides in the gap. Where such nomenclature is followed by a specific modification, that modification is the modification in each sugar moiety of each wing and the gap nucleosides comprise a 2′-deoxyribosyl sugar moiety. Thus, a 5-10-5 MOE gapmer consists of 5 linked 2′-MOE nucleosides in the 5′-wing, 10 linked 2′-deoxyribonucleosides in the gap, and 5 linked 2′-MOE nucleosides in the 3′-wing.

In certain embodiments, each nucleoside of a modified oligonucleotide, or portion thereof, comprises a 2′-substituted sugar moiety, a bicyclic sugar moiety, a sugar surrogate, or a 2′-deoxyribosyl sugar moiety. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, PNA, THP, and F-HNA.

In certain embodiments, modified oligonucleotides comprise at least 12, at least 13, at least 14, at least 15, at least 16, at least 17, at least 18, at least 19, or at least 20 nucleosides comprising a modified sugar moiety. In certain embodiments, the modified sugar moiety is selected independently from a 2′-substituted sugar moiety, a bicyclic sugar moiety, or a sugar surrogate. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA.

In certain embodiments, each nucleoside of a modified oligonucleotide comprises a modified sugar moiety (“fully modified oligonucleotide”). In certain embodiments, each nucleoside of a fully modified oligonucleotide comprises a 2′-substituted sugar moiety, a bicyclic sugar moiety, or a sugar surrogate. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA. In certain embodiments, each nucleoside of a fully modified oligonucleotide comprises the same modified sugar moiety (“uniformly modified sugar motif”). In certain embodiments, the uniformly modified sugar motif is 7 to 20 nucleosides in length. In certain embodiments, each nucleoside of the uniformly modified sugar motif comprises a 2′-substituted sugar moiety, a bicyclic sugar moiety, or a sugar surrogate. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA. In certain embodiments, modified oligonucleotides having at least one fully modified sugar motif may also comprise at least 1, at least 2, at least 3, or at least 4 2′-deoxyribonucleosides.

2. Certain Nucleobase Motifs

In certain embodiments, oligonucleotides comprise modified and/or unmodified nucleobases arranged along the oligonucleotide, or portion thereof, in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methyl cytosines. In certain embodiments, all of the cytosine nucleobases are 5-methyl cytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of the nucleoside is a 2′-deoxyribosyl sugar moiety. In certain embodiments, the modified nucleobase is selected from: a 2-thiopyrimidine and a 5-propynepyrimidine.

3. Certain Internucleoside Linkage Motifs

In certain embodiments, oligonucleotides comprise modified and/or unmodified internucleoside linkages arranged along the oligonucleotide, or portion thereof, in a defined pattern or motif. In certain embodiments, each internucleoside linking group is a phosphodiester internucleoside linkage. In certain embodiments, each internucleoside linking group of a modified oligonucleotide is a phosphorothioate internucleoside linkage. In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate, a (Sp) phosphorothioate, and a (Rp) phosphorothioate. In certain embodiments, the sugar motif of a modified oligonucleotide is a gapmer and the internucleoside linkages within the gap are all modified. In certain such embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester internucleoside linkage is not a terminal internucleoside linkage, and the remaining internucleoside linkages are phosphorothioate internucleoside linkages. In certain such embodiments, all of the phosphorothioate internucleoside linkages are stereorandom. In certain embodiments, all of the phosphorothioate internucleoside linkages in the wings are (Sp) phosphorothioates, and the gap comprises at least one Sp, Sp, Rp motif. In certain embodiments, populations of modified oligonucleotides are enriched for modified oligonucleotides comprising such internucleoside linkage motifs.

In certain embodiments, modified oligonucleotides comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 phosphodiester internucleoside linkages. In certain embodiments, modified oligonucleotides comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 phosphorothioate internucleoside linkages. In certain embodiments, modified oligonucleotides comprise at least 1, at least 2, at least 3, at least 4, or at least 5 phosphodiester internucleoside linkages and the remainder of the internucleoside linkages are phosphorothioate internucleoside linkages.

B. Certain Lengths

It is possible to increase or decrease the length of an oligonucleotide without eliminating activity. For example, in Woolf et al., Proc. Natl. Acad. Sci. USA, 1992, 89, 7305-7309, 1992), a series of oligonucleotides 13-25 nucleobases in length were tested for their ability to induce cleavage of a target nucleic acid in an oocyte injection model. Oligonucleotides 25 nucleobases in length with 8 or 11 mismatch bases near the ends of the oligonucleotides were able to direct specific cleavage of the target nucleic acid, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 27, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments, oligonucleotides consist of 16 linked nucleosides. In certain embodiments, oligonucleotides consist of 17 linked nucleosides. In certain embodiments, oligonucleotides consist of 18 linked nucleosides. In certain embodiments, oligonucleotides consist of 19 linked nucleosides. In certain embodiments, oligonucleotides consist of 20 linked nucleosides.

C. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

D. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular stereochemical configuration.

E. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a portion of an oligonucleotide has a nucleobase sequence that is complementary to a second oligonucleotide or an identified reference nucleic acid, such as a target nucleic acid. In certain embodiments, the nucleobase sequence of a portion or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

### III. Certain Oligomeric Compounds

In certain embodiments, provided herein are oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, abasic nucleosides, modified or unmodified nucleosides, and two or more nucleosides that are independently modified or unmodified.

A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge, and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

1. Conjugate Moieties

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, lipophilic groups, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial, or an antibiotic.

2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain oligomeric compounds, a conjugate moiety is attached to an oligonucleotide via a more complex conjugate linker comprising one or more conjugate linker moieties, which are sub-units making up a conjugate linker. In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxyribonucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate internucleoside linkage. In certain such embodiments, the cleavable moiety is 2′-deoxyadenosine.

B. Certain Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphonates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

C. Oligomeric Duplexes

In certain embodiments, oligomeric compounds described herein comprise an oligonucleotide, having a nucleobase sequence complementary to that of a target nucleic acid. In certain embodiments, an oligomeric compound is paired with a second oligomeric compound to form an oligomeric duplex. Such oligomeric duplexes comprise a first oligomeric compound having a portion complementary to a target nucleic acid and a second oligomeric compound having a portion complementary to the first oligomeric compound. In certain embodiments, the first oligomeric compound of an oligomeric duplex comprises or consists of (1) a modified or unmodified oligonucleotide and optionally a conjugate group and (2) a second modified or unmodified oligonucleotide and optionally a conjugate group. Either or both oligomeric compounds of an oligomeric duplex may comprise a conjugate group. The oligonucleotides of each oligomeric compound of an oligomeric duplex may include non-complementary overhanging nucleosides.

D. Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they reduce, modulate, or increase the amount or activity of a target nucleic acid by 25% or more in the standard cell assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, provided herein are antisense compounds that are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid. In certain embodiments, hybridization of an antisense compound to a target nucleic acid results in exon inclusion. In certain embodiments, hybridization of an antisense compound to a target nucleic acid results in an increase in the amount or activity of a target nucleic acid. In certain embodiments, hybridization of an antisense compound complementary to a target nucleic acid results in alteration of splicing, leading to the inclusion of an exon in the mRNA.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or subject.

### IV. Certain Target Nucleic Acids

In certain embodiments, oligomeric compounds comprise or consist of an oligonucleotide comprising a portion that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target nucleic acid is a mature mRNA. In certain embodiments, the target nucleic acid is a pre-mRNA. In certain embodiments, the target region is entirely within an intron. In certain embodiments, the target region spans an intron/exon junction. In certain embodiments, the target region is at least 50% within an intron.

A. Complementarity/Mismatches to the Target Nucleic Acid

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and a 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a portion that is 100% or fully complementary to a target nucleic acid. In certain embodiments, the portion of full complementarity is 6, 7, 8, 9, 10, 11, 12, 13, 14, 15, 16, 17, 18, 19, or 20 nucleobases in length.

In certain embodiments, oligonucleotides comprise one or more mismatched nucleobases relative to the target nucleic acid. In certain embodiments, the mismatch is at position 1, 2, 3, 4, 5, 6, 7, 8, 9, 10, 11, 12, 13, 14, 15, 16, 17, 18, 19, or 20 from the 5′-end of the oligonucleotide.

### V. Certain Pharmaceutical Compositions

Oligomeric compounds described herein may be admixed with pharmaceutically acceptable active or inert substances for the preparation of pharmaceutical compositions. Compositions and methods for the formulation of pharmaceutical compositions are dependent upon a number of criteria, including, but not limited to, route of administration, extent of disease, or dose to be administered.

Certain embodiments provide pharmaceutical compositions comprising one or more oligomeric compounds or a salt thereof. In certain embodiments, the oligomeric compounds comprise or consist of a modified oligonucleotide. In certain such embodiments, the pharmaceutical composition comprises a suitable pharmaceutically acceptable diluent or carrier. In certain embodiments, a pharmaceutical composition comprises a sterile saline solution and one or more oligomeric compound. In certain embodiments, such pharmaceutical composition consists of a sterile saline solution and one or more oligomeric compound. In certain embodiments, the sterile saline is pharmaceutical grade saline. In certain embodiments, a pharmaceutical composition comprises one or more oligomeric compound and sterile water. In certain embodiments, a pharmaceutical composition consists of one oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, a pharmaceutical composition consists of one or more oligomeric compound and sterile PBS. In certain embodiments, the sterile PBS is pharmaceutical grade PBS. Compositions and methods for the formulation of pharmaceutical compositions are dependent upon a number of criteria, including, but not limited to, route of administration, extent of disease, or dose to be administered.

An oligomeric compound described herein complementary to a target nucleic acid can be utilized in pharmaceutical compositions by combining the oligomeric compound with a suitable pharmaceutically acceptable diluent or carrier and/or additional components such that the pharmaceutical composition is suitable for injection. In certain embodiments, a pharmaceutically acceptable diluent is phosphate buffered saline. Accordingly, in one embodiment, employed in the methods described herein is a pharmaceutical composition comprising an oligomeric compound complementary to a target nucleic acid and a pharmaceutically acceptable diluent. In certain embodiments, the pharmaceutically acceptable diluent is phosphate buffered saline. In certain embodiments, the oligomeric compound comprises or consists of a modified oligonucleotide provided herein.

Pharmaceutical compositions comprising oligomeric compounds provided herein encompass any pharmaceutically acceptable salts, esters, or salts of such esters, or any other oligonucleotide which, upon administration to an animal, including a human, is capable of providing (directly or indirectly) the biologically active metabolite or residue thereof. In certain embodiments, the oligomeric compound comprises or consists of a modified oligonucleotide. Accordingly, for example, the disclosure is also drawn to pharmaceutically acceptable salts of compounds, prodrugs, pharmaceutically acceptable salts of such prodrugs, and other bioequivalents. Suitable pharmaceutically acceptable salts include, but are not limited to, sodium and potassium salts.

### VI. Certain Mechanisms

In certain embodiments, oligomeric compounds described herein comprise or consist of modified oligonucleotides having at least one stereo-non-standard nucleoside. In certain such embodiments, the oligomeric compounds described herein are capable of hybridizing to a target nucleic acid, resulting in at least one antisense activity. In certain embodiments, compounds described herein selectively affect one or more target nucleic acid. Such compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in a significant undesired antisense activity.

In certain antisense activities, hybridization of a compound described herein to a target nucleic acid results in recruitment of a protein that cleaves the target nucleic acid. For example, certain compounds described herein result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, compounds described herein are sufficiently “DNA-like” to elicit RNase H activity. Further, in certain embodiments, one or more non-DNA-like nucleoside in in the RNA:DNA duplex is tolerated.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or animal.

### Nonlimiting Disclosure and Incorporation by Reference

Each of the literature and patent publications listed herein is incorporated by reference in its entirety. While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar moiety (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as a or R such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, all cis- and trans-isomers and tautomeric forms of the compounds herein are also included unless otherwise indicated. Oligomeric compounds described herein include chirally pure or enriched mixtures as well as racemic mixtures. For example, oligomeric compounds having a plurality of phosphorothioate internucleoside linkages include such compounds in which chirality of the phosphorothioate internucleoside linkages is controlled or is random. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif And, for example, where a particular high-affinity modification appears at a particular position, other high-affinity modifications at the same position are considered suitable, unless otherwise indicated.

### Example 1. Synthesis of Backbone Constrained Macrocycle, Compound 6.24

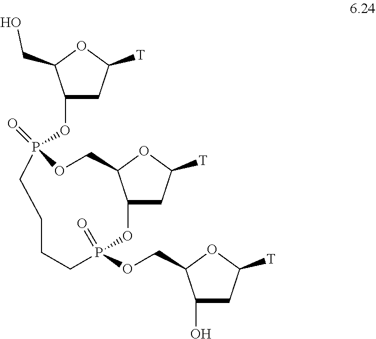

### Preparation of Compound 6.10

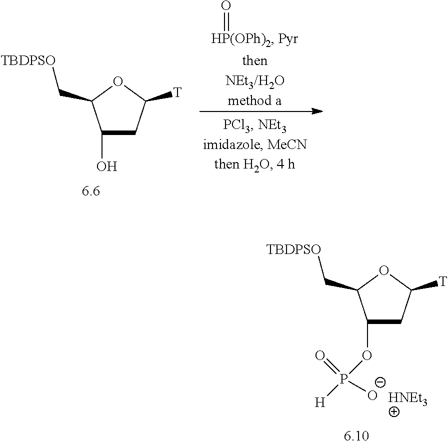

### Method a

To a stirred solution of compound 6.6 (1.3065 g, 2.71 mmol) in pyridine (14 mL) was added diphenyl phosphite (3.6 mL, 18.8 mmol). After 15 min, a 1:1 mixture of triethylamine:water (6 mL) was added and the resulting mixture was stirred for 15 min after which the solvent was removed under reduced pressure. The crude residue was dissolved in CH₂Cl₂ (150 mL) and washed with aqueous 5% NaHCO₃ (3×60 mL), dried over Na₂SO₄, filtered and concentrated under reduced pressure. The residue was purified by flash chromatography (10% MeOH in CH₂Cl₂+1% NEt₃) to give nucleoside 6.10 (1.423 mg, 81%) Rf=0.31 (10% MeOH in CH₂Cl₂+1% NEt₃).

### Method b

Phosphorous trichloride (0.289 mL, 3.31 mmol) and triethylamine (1.38 mL, 9.92 mmol) were added sequentially to a stirred solution of imidazole (675 mg, 9.92 mmol) in acetonitrile (20 mL) at 0° C. After stirring for 30 min, a solution of compound 6.6 (318 mg, 0.661 mmol) in acetonitrile (15 mL) was added, the ice bath was removed and the reaction mixture was allowed to react at room temperature for 3 h. Water (6.2 mL) was added to the reaction mixture and stirred for 30 min then the solvent was removed under reduced pressure. The crude residue was dissolved in a mixture containing pyridine (5 mL) and triethylamine (1 mL), and then evaporated to dryness. The dry residue was partitioned between water and CH₂Cl₂, the layers were separated, and the aqueous layer was extracted with CH₂Cl₂ (3×20 mL). The combined organic extracts were dried over Na₂SO₄, filtered, and concentrated under reduced pressure. The residue was purified by flash chromatography (10% MeOH in CH₂Cl₂+1% NEt₃) to give nucleoside 6.10 (385 mg, 83%) Rf=0.31 (10% MeOH in CH₂Cl₂+1% NEt₃); ¹H NMR (400 MHz, CDCl3) δ 7.65 (s, 1H, J=617.2 Hz, H-P), 7.60 (td, J=7.6, 7.0, 1.7 Hz, 4H), 7.42-7.26 (m, 7H), 6.39 (dd, J=8.8, 5.3 Hz, 1H), 6.11 (s, J=617.2 Hz, 1H H-P), 4.95 (td, J=7.2, 2.3 Hz, 1H), 4.21-4.15 (m, 1H), 3.99-3.83 (m, 2H), 3.00 (q, J=7.3 Hz, 7H), 2.50 (ddd, J=13.3, 5.3, 1.9 Hz, 1H), 2.22-2.12 (m, 1H), 1.40 (s, 3H), 1.26 (t, J=7.3 Hz, 11H), 1.02 (s, 9H); ³¹P NMR (162 MHz, CDCl₃) δ 3.53; ¹³C NMR (101 MHz, CDCl₃) δ 164.13, 150.73, 135.50, 135.23, 135.19, 133.16, 132.33, 129.98, 129.87, 127.93, 127.85, 111.08, 86.16, 86.10, 84.30, 73.75, 73.71, 64.07, 45.54, 39.70, 39.67, 26.99, 19.34, 11.83, 8.62; ³¹P NMR (162 MHz, CDCl₃) δ 3.53.

### Preparation of Compound 6.13

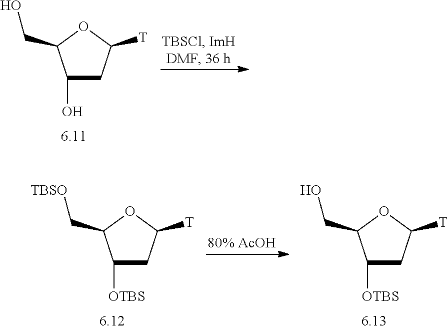

Imidazole (10.2 g, 0.15 mol) and TBSCl (17.8 mL, 0.125 mol) were added to a stirred solution of thymidine 6.11 (12.1 g, 0.05 mol) in pyridine (200 mL). After stirring overnight, pyridine was evaporated and the residue was diluted with ethyl acetate, washed with brine (×3), dried over Na₂SO₄, filtered, and concentrated under reduced pressure to give crude 6.12. The residue was dissolved in 80% AcOH (300 mL) and the resulting solution stood at room temperature for 4 days. Solvents were evaporated and the residue was passed through a short silica column (3:7 EtOAc/hex) to give 11.3 g (63%) of 6.13 as a white solid. The ¹H and ¹³C NMR matches with the reported data.²¹⁹

### Preparation of Compounds 6.14 and 6.15

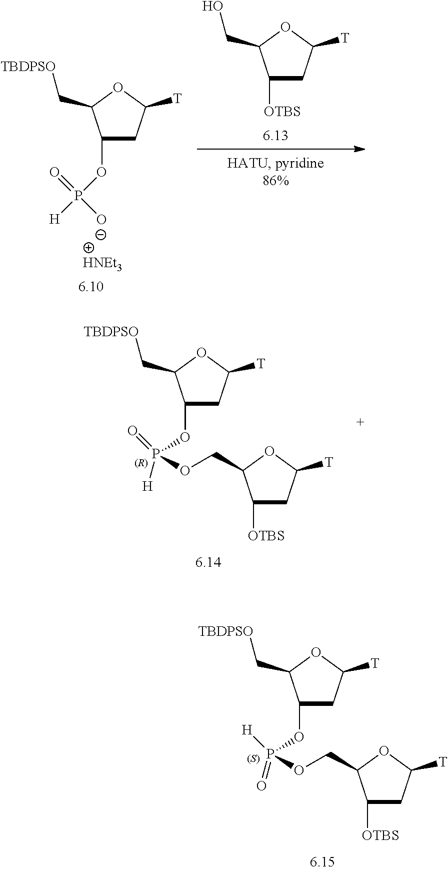

Compounds 6.10 (2.0 g, 3.1 mmol) and 6.13 (1.66 g, 4.65 mmol) were mixed in a flask with anhydrous pyridine (5 mL) and evaporated to dryness. The dry residue was dissolved in anhydrous pyridine (40 mL) and HATU (2.4 g, 6.19 mmol) was added. The reaction mixture was stirred for 3 h then the solvent was removed under reduced pressure. The dry residue was dissolved in EtOAc (50 mL) and a white precipitate formed was filtered off. The organic later was washed with 5% LiCl (5×5 mL). The organic extract was dried over Na₂SO₄, filtered, and concentrated under reduced pressure. The residue was purified by flash chromatography to give nucleoside 6.14 (1.2 g), 6.15 (0.50 g) and 0.5 g of a mixture containing 6.14 and 6.15.

6.14: ¹H NMR (500 MHz, CDCl₃) δ 10.25 (s, 1H), 10.19 (s, 1H), 7.60 (t, J=7.7 Hz, 5H), 7.43-7.29 (m, 9H), 6.38 (dd, J=8.9, 5.5 Hz, 1H), 6.21-6.17 (m, 1H), 5.25 (t, J=6.8 Hz, 1H), 4.44-4.31 (m, 2H), 4.27-4.13 (m, 2H), 4.00-3.90 (m, 2H), 3.90-3.82 (m, 1H), 2.75 (s, 2H), 2.58 (dd, J=14.1, 5.4 Hz, 1H), 2.34-2.19 (m, 3H), 1.87 (s, 3H), 1.53 (s, 3H), 1.04 (s, 9H), 0.85 (s, 9H), 0.05 (s, 6H); ¹³C NMR (126 MHz, CDCl₃) δ 164.19, 164.04, 150.64, 150.48, 135.89, 135.43, 135.39, 135.16, 135.09, 134.60, 132.52, 131.85, 130.17, 130.04, 127.99, 127.92, 127.85, 127.81, 111.54, 111.10, 85.60, 85.25, 85.20, 84.64, 84.58, 84.06, 77.36, 71.12, 64.71, 64.66, 63.58, 40.19, 39.26, 38.50, 26.89, 25.64, 25.59, 19.25, 19.21, 17.77, 12.36, 11.95, 11.91, −4.74, −4.96; ³¹P NMR (202 MHz, CDCl₃) δ 7.36; HRMS (ESI) calc'd for C₄₂H₆₀N₄O₁₁PSi₂ [M+H]⁺ m/z=883.35292, found 833.35502.

6.15: ¹H NMR (400 MHz, CDCl₃) δ 9.14 (d, J=24.1 Hz, 2H), 7.84 (d, J=2.4 Hz, 1H), 7.64 (td, J=8.2, 1.6 Hz, 4H), 7.49-7.35 (m, 7H), 7.30 (s, 1H), 6.41 (dd, J=9.1, 5.2 Hz, 1H), 6.22 (t, J=6.6 Hz, 1H), 6.05 (s, 1H), 5.24 (t, J=7.0 Hz, 1H), 4.38-4.31 (m, 1H), 4.30-4.14 (m, 3H), 4.04-3.88 (m, 3H), 2.56 (dd, J=13.9, 5.3 Hz, 1H), 2.33-2.23 (m, 2H), 2.23-2.13 (m, 1H), 1.92 (d, J=1.2 Hz, 3H), 1.57 (s, 3H), 1.09 (s, 9H), 0.88 (d, J=2.8 Hz, 10H), 0.08 (t, J=2.2 Hz, 6H); ¹³C NMR (101 MHz, CDCl₃) δ 163.75, 150.57, 150.38, 135.83, 135.64, 135.34, 134.82, 132.75, 132.13, 130.44, 130.33, 128.26, 128.19, 111.84, 111.59, 85.86, 85.67, 85.02, 84.39, 71.35, 63.75, 40.58, 27.15, 25.79, 19.51, 18.02, 12.62, 12.12, −4.51, −4.73; ³¹P NMR (162 MHz, CDCl₃) δ 8.66; HRMS (ESI) calc'd for C₄₂H₆₀N₄O₁₁PSi₂ [M+H]⁺ m/z=883.35292, found 833.35526.

### Preparation of Compound 6.16

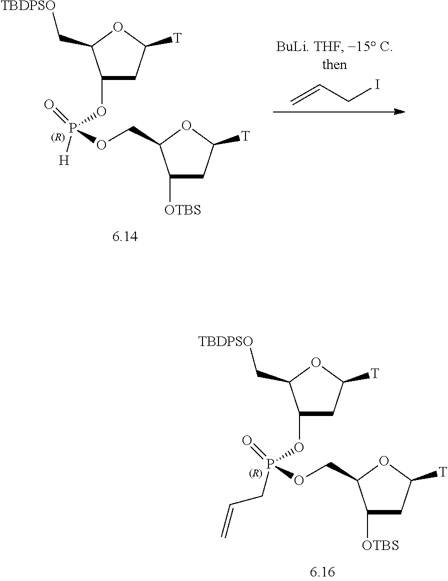

To a stirred solution of compound 6.14 (800 mg, 0.906 mmol) in THF (150 mL) at −15° C. was added dropwise a solution of 2.5 M BuLi in hexanes (1.3 mL, 3.25 mmol). After 5 min, allyl iodide (0.38 mL, 4.15 mmol) was added dropwise and the reaction mixture was kept at −15° C. for 30 min, followed by removal of the solvent aided by a rotavap system warmed at 35° C. The crude residue was dissolved in EtOAc (200 mL), washed with water and brine. The combined organic extractions were dried over Na₂SO₄, filtered, and concentrated under reduced pressure. The residue was purified by flash chromatography (8:2 ethyl acetate/hexane) to give compound 6.16 as a white foam (553 mg, 66%); ¹H NMR (500 MHz, CDCl₃) δ 10.18 (s, 1H), 10.12 (s, 1H), 7.65-7.56 (m, 4H), 7.43-7.31 (m, 8H), 6.38 (dd, J=9.1, 5.3 Hz, 1H), 6.21 (t, J=6.7 Hz, 1H), 5.70 (ddq, J=17.2, 10.1, 7.2 Hz, 1H), 5.23-5.09 (m, 3H), 4.36 (dt, J=7.1, 3.7 Hz, 1H), 4.31-4.23 (m, 1H), 4.20-4.11 (m, 2H), 4.00 (q, J=4.0 Hz, 1H), 3.94 (dd, J=11.6, 2.6 Hz, 1H), 3.82 (dd, J=11.7, 2.6 Hz, 1H), 2.69-2.59 (m, 2H), 2.54 (dd, J=13.5, 5.3 Hz, 1H), 2.30-2.19 (m, 2H), 2.12 (dt, J=13.5, 6.8 Hz, 1H), 1.89 (d, J=1.2 Hz, 3H), 1.53 (s, 3H), 1.04 (s, 9H), 0.85 (s, 10H), 0.05 (s, 6H); ¹³C NMR (126 MHz, CDCl₃) δ 164.26, 164.07, 150.72, 150.53, 135.69, 135.46, 135.16, 134.67, 132.63, 131.95, 130.19, 130.08, 128.03, 127.96, 126.32, 126.23, 121.13, 121.01, 111.59, 111.10, 85.58, 85.53, 85.48, 85.07, 85.01, 84.13, 77.36, 77.09, 77.04, 71.48, 65.01, 64.96, 63.84, 40.47, 39.49, 32.39, 31.29, 26.94, 25.64, 19.28, 17.85, 12.38, 11.95, −4.68, −4.88; ³¹P NMR (202 MHz, CDCl₃) δ 27.88; HRMS (ESI) calc'd for C₄₅H₆₄N₄O₁₁PSi₂ [M+H]⁺ m/z=923.38422, found 923.38536.

### Preparation of Compound 6.17

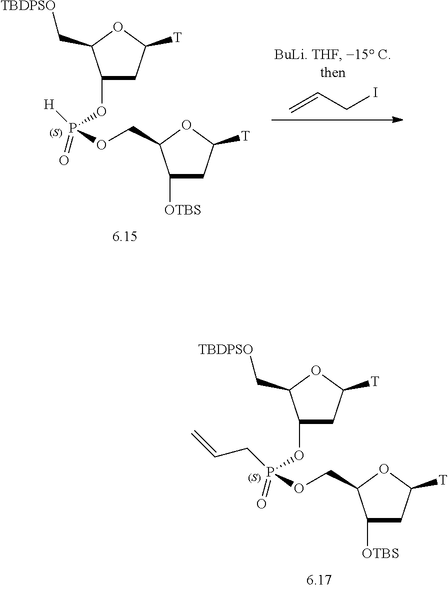

To a stirred solution of compound 6.15 (100 mg, 0.113 mmol) in THF (20 mL) at −15° C. was added dropwise a solution of 2.5 M BuLi in hexanes (0.16 mL, 0.39 mmol). After 5 min, allyl iodide (50 μL, 0.50 mmol) was added dropwise and the reaction mixture was kept at −15° C. for 30 min, followed by removal of the solvent aided by a rotavap system warmed at 35° C. The crude residue was dissolved in EtOAc (20 mL), washed with water and brine. The combined organic extractions were dried over Na₂SO₄, filtered, and concentrated under reduced pressure. The residue was purified by flash chromatography (8:2 ethyl acetate/hexane) to give compound 6.17 as a white foam (65 mg, %); ¹H NMR (300 MHz, CDCl₃) δ 8.57 (d, J=15.4 Hz, 2H), 7.69-7.60 (m, 4H), 7.49-7.36 (m, 8H), 7.30-7.27 (m, 1H), 6.38 (dd, J=9.2, 5.2 Hz, 1H), 6.20 (t, J=6.7 Hz, 1H), 5.89-5.67 (m, 1H), 5.33-5.14 (m, 3H), 4.30 (dt, J=6.7, 3.5 Hz, 1H), 4.26-4.10 (m, 2H), 4.10-3.81 (m, 4H), 2.69 (ddd, J=22.0, 7.5, 1.4 Hz, 2H), 2.52 (dd, J=13.7, 5.3 Hz, 1H), 2.34-2.17 (m, 2H), 2.17-2.00 (m, 1H), 1.99-1.89 (m, 3H), 1.57 (d, J=1.2 Hz, 3H), 1.09 (s, 9H), 0.88 (s, 9H), 0.08-0.04 (m, 6H); ¹³C NMR (101 MHz, CDCl₃) δ 163.84, 163.80, 150.58, 150.34, 135.66, 135.54, 135.35, 134.86, 132.77, 132.19, 130.38, 130.30, 128.22, 128.17, 126.44, 126.33, 121.46, 121.31, 111.73, 111.37, 86.00, 85.96, 85.62, 85.38, 85.32, 84.41, 77.05, 71.68, 65.10, 65.04, 63.93, 60.51, 40.75, 39.57, 39.52, 32.74, 31.36, 27.14, 25.79, 21.16, 19.51, 18.02, 14.32, 12.69, 12.64, 12.12, −4.53, −4.55, −4.72, −4.75; ³¹P NMR (121 MHz, CDCl₃) δ 28.65.

### Preparation of Compound 6.18

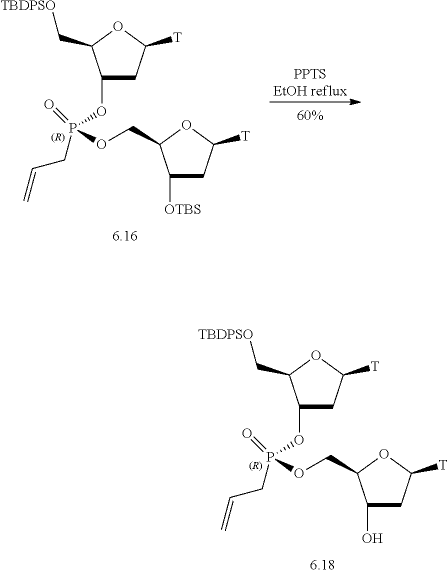

To a stirred solution of compound 6.16 (553 mg, 0.599 mmol) in ethanol (3 mL) was added PPTS (301 mg, 1.19 mmol) and the resulting solution was heated to reflux for 36 h. The reaction mixture was cooled to room temperature and combined with NaHCO₃ (sat) and CH₂Cl₂. The aqueous layer was extracted with CH₂Cl₂ (3×50 mL). The combined organic extractions were dried over Na₂SO₄, filtered, and concentrated under reduced pressure. The residue was purified by flash chromatography (CH₂Cl₂ neat to 6% MeOH in CH₂Cl₂) to give compound 6.18 as a white foam (290 mg, 60%); ¹H NMR (400 MHz, CDCl₃) δ 10.34 (s, 1H), 9.96 (s, 1H), 7.70-7.59 (m, 4H), 7.50-7.34 (m, 8H), 6.34 (dd, J=9.4, 4.9 Hz, 1H), 6.25 (t, J=6.6 Hz, 1H), 5.70 (ddt, J=17.0, 9.9, 7.2 Hz, 1H), 5.26-5.07 (m, 3H), 4.64-4.22 (m, 4H), 4.19-4.08 (m, 2H), 3.94 (dd, J=11.8, 2.8 Hz, 1H), 3.79 (dd, J=11.8, 2.6 Hz, 1H), 2.75-2.55 (m, 3H), 2.51-2.40 (m, 1H), 2.36-2.11 (m, 3H), 1.90 (s, 3H), 1.59 (s, 3H), 1.07 (s, 9H); ¹³C NMR (101 MHz, CDCl₃) δ 164.33, 164.08, 151.32, 150.79, 135.92, 135.66, 135.38, 134.69, 132.65, 132.08, 130.43, 130.32, 128.24, 128.18, 126.40, 126.29, 121.41, 121.26, 112.07, 111.16, 85.66, 84.91, 84.59, 78.04, 71.05, 65.52, 64.12, 40.29, 40.08, 32.80, 31.42, 27.11, 19.42, 12.64, 12.20; ³¹P NMR (162 MHz, CDCl₃) δ 28.28. HRMS (ESI) calc'd for C₃₉H₅₀N₄O₁₁PSi [M+H]⁺ m/z=809.2977, found 809.2984.

### Preparation of Compound 6.19

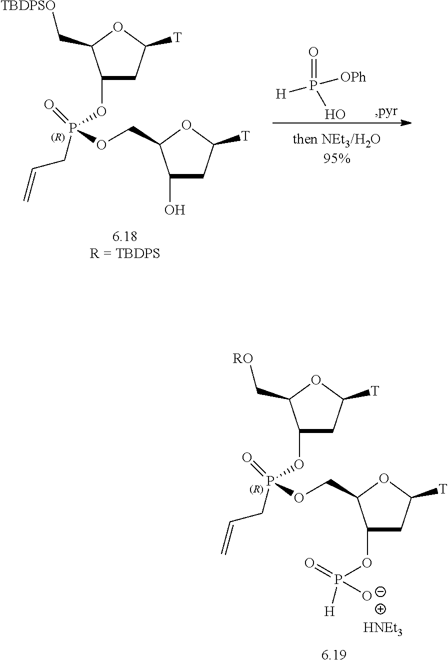

To a stirred solution of compound 6.18 (290 mg, 0.358 mmol) in pyridine (1.8 mL) was added diphenyl phosphite (0.48 mL, 2.50 mmol). After 15 min, a 1:1 mixture of triethylamine:water (2 mL) was added and the resulting mixture was stirred for 15 min after which the solvent was removed under reduced pressure. The crude residue was dissolved in CH₂Cl₂ (20 mL) and washed with aqueous 5% NaHCO₃ (3×5 mL), dried over Na₂SO₄, filtered and concentrated under reduced pressure. The residue was purified by flash chromatography (10% MeOH in CH₂Cl₂+1% NEt₃) to give nucleoside 6.19 (330 mg, 95%) Rf=(10% MeOH in CH₂Cl₂+1% NEt₃); ¹H NMR (400 MHz, CDCl₃) δ 7.63 (td, J=8.0, 1.6 Hz, 4H), 7.45-7.34 (m, 8H), 6.40-6.23 (m, 2H), 5.70 (ddq, J=17.1, 9.9, 7.2 Hz, 1H), 5.23-5.08 (m, 3H), 4.86 (s, 1H), 4.32 (q, J=9.8 Hz, 3H), 4.12 (q, J=2.3 Hz, 1H), 3.94 (dd, J=11.6, 2.6 Hz, 1H), 3.81 (dd, J=11.8, 2.7 Hz, 1H), 3.55 (qd, J=9.4, 8.3, 4.8 Hz, 2H), 3.07 (q, J=7.3 Hz, 8H), 2.70-2.45 (m, 4H), 2.31-2.08 (m, 2H), 1.92 (s, 3H), 1.50 (s, 4H), 1.34 (t, J=7.3 Hz, 16H), 1.07 (s, 9H); ¹³C NMR (126 MHz, CDCl₃) δ 164.08, 163.97, 150.50, 150.42, 135.21, 134.93, 134.53, 132.44, 131.77, 129.93, 129.80, 129.00, 128.71, 127.91, 127.78, 127.70, 126.14, 126.05, 124.98, 122.88, 120.86, 120.75, 120.41, 120.37, 111.08, 110.90, 85.15, 85.10, 84.59, 83.81, 83.72, 77.36, 72.51, 65.34, 65.28, 63.49, 52.39, 45.41, 39.04, 38.86, 34.01, 32.09, 30.99, 26.70, 21.58, 21.16, 19.02, 14.50, 12.13, 11.74, 8.36, 7.48; ³¹P NMR (202 MHz, CDCl₃) δ 27.82, 2.82, 0.39. HRMS (ESI) calc'd for C₃₉H₅₁N₄O₁₃P₂Si [M+H]⁺ m/z=873.2692, found 873.2687.

### Preparation of Compounds 6.20 and 6.21

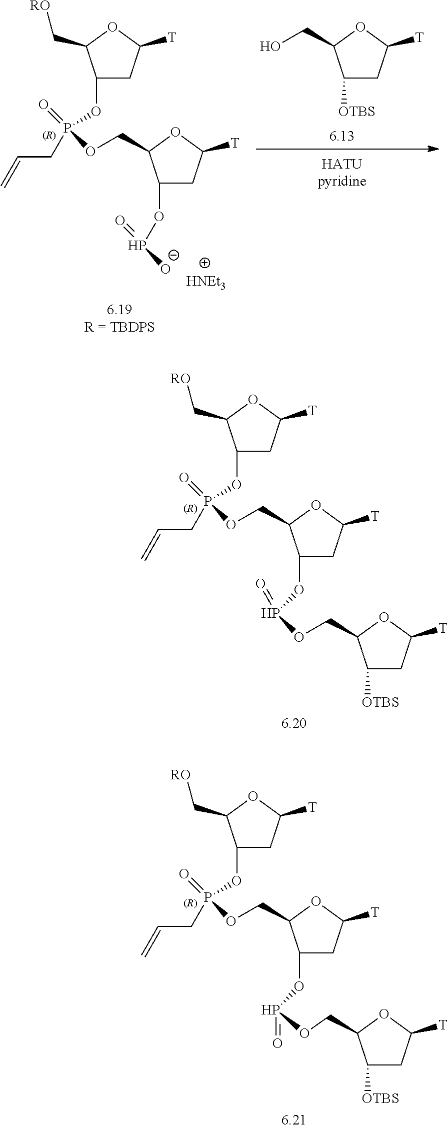

Compounds 6.19 (330 mg, 0.34 mmol) and 6.13 (181 mg, 0.50 mmol) were mixed in a flask with anhydrous pyridine (1 mL) and evaporated to dryness. The dry residue was dissolved in anhydrous pyridine (1.7 mL) and HATU (260 mg) was added. The reaction mixture was stirred for 3.5 h then the solvent was removed under reduced pressure. The dry residue was dissolved in EtOAc (50 mL) and a white precipitate formed was filtered off. The organic later was washed with 5% LiCl (5×5 mL). The organic extract was dried over Na₂SO₄, filtered and concentrated under reduced pressure. The residue was purified by flash chromatography (0% to 5% MeOH in CH₂Cl₂) to give nucleoside 6.20 (83 mg, 20%), and a mixture of 6.20 and 6.21 (117 mg, 28%). 6.20: ¹H NMR (400 MHz, CDCl₃) δ 9.60 (s, 1H), 9.39 (s, 1H), 9.02 (s, 1H), (7.85 and 5.30, d, 1021.2 Hz), 7.64 (ddd, J=8.0, 5.1, 1.6 Hz, 4H), 7.50-7.31 (m, 8H), 6.33 (dd, J=9.4, 4.9 Hz, 1H), 6.20 (dd, J=7.7, 6.1 Hz, 1H), 6.09-6.01 (m, 1H), 5.79-5.65 (m, 1H), 5.25-5.10 (m, 4H), 4.49-4.19 (m, 6H), 4.14 (d, J=4.0 Hz, 1H), 4.01 (q, J=4.3 Hz, 1H), 3.94 (dd, J=11.6, 2.9 Hz, 1H), 3.81 (ddd, J=11.7, 8.8, 2.0 Hz, 1H), 2.76-2.14 (m, 8H), 1.96-1.86 (m, 6H), 1.63-1.57 (m, 3H), 1.08 (s, 9H), 0.89 (d, J=1.9 Hz, 10H), 0.09 (s, 6H); ³¹P NMR (162 MHz, CDCl₃) δ 27.95, 7.48. 6.21: ³¹P NMR (162 MHz, CDCl₃) δ 27.70, 8.27.

### Preparation of Compound 6.22

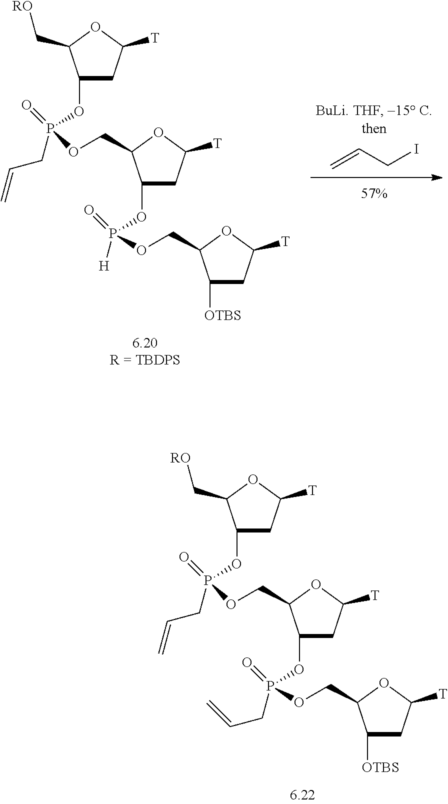

To a stirred solution of compound 6.20 (83 mg, 68 mol) in THF (2 mL) at −15° C. was added dropwise a solution of 2.5 M BuLi in hexanes (0.13 mL, 0.325 mmol). After 5 min, allyl iodide (30 μL, 0.33 mmol) was added dropwise and the reaction mixture was kept at −15° C. for 30 min, followed by removal of the solvent aided by a rotavap system warmed at 35° C. The crude residue was dissolved in EtOAc (20 mL), washed with water and brine. The combined organic extractions were dried over Na₂SO₄, filtered and concentrated under reduced pressure. The residue was purified by flash chromatography (8:2 ethyl acetate/hexane) to give compound 6.22 as a white foam (49 mg, 57%); ¹H NMR (400 MHz, CDCl₃) δ 9.82-9.57 (m, 3H), 7.64 (ddd, J=8.0, 6.3, 1.7 Hz, 4H), 7.48-7.34 (m, 9H), 6.34 (dd, J=9.2, 5.1 Hz, 1H), 6.20 (t, J=6.8 Hz, 2H), 5.84-5.61 (m, 2H), 5.32-5.05 (m, 6H), 4.43-4.08 (m, 7H), 4.01 (dd, J=5.3, 3.4 Hz, 1H), 3.93 (dd, J=11.7, 2.8 Hz, 1H), 3.80 (dd, J=11.6, 2.8 Hz, 1H), 2.76-2.45 (m, 6H), 2.24 (dt, J=12.5, 6.7 Hz, 4H), 1.97-1.84 (m, 6H), 1.07 (s, 9H), 0.87 (s, 9H), 0.07 (s, 6H); ¹³C NMR (101 MHz, CDCl₃) δ 164.16, 164.02, 163.96, 150.73, 150.67, 150.54, 136.36, 135.78, 135.66, 135.38, 134.82, 132.76, 132.18, 130.36, 130.25, 128.20, 128.14, 126.42, 126.36, 121.48, 121.33, 121.25, 111.67, 111.61, 111.42, 86.20, 85.83, 85.65, 85.24, 84.42, 83.86, 75.79, 71.72, 65.71, 65.06, 64.02, 40.15, 39.66, 32.75, 31.37, 27.11, 25.80, 19.44, 18.02, 12.47, 12.15, −4.53, −4.73; ³¹P NMR (162 MHz, CDCl₃) δ 28.09, 27.85; HRMS (ESI) calc'd for C₅₈H₈₁N₆O₁₇P₂Si₂ [M+H]⁺ m/z=1251.4666, found 1251.46610.

### Preparation of Compound 6.23

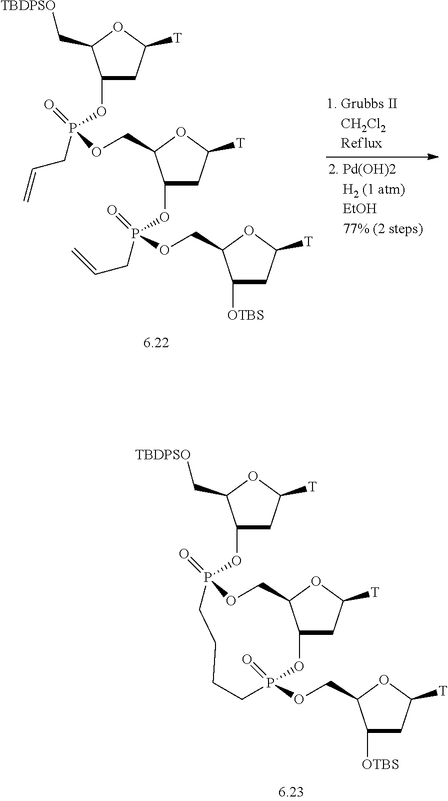

Second generation Grubbs catalyst (3.3 mg, 3.9 mol) was added to a stirred solution of compound 6.22 (49 mg, 39 mol) in CH₂Cl₂ (2 mL). After heating the reaction to reflux for 5 h, silica gel was added to the mixture and the solvent was removed under reduced pressure. The dry residue was purified through a short chromatographic column using 8% MeOH in CH₂Cl₂ obtaining an inseparable cis trans mixture of alkenes (41 mg). The olefin mixture was dissolved in ethanol (0.5 mL) and mixed with Pd(OH)₂. (2.3 mg, 3.3 mol) The flask was degassed under vacuum and refilled with hydrogen (×3). A balloon filled with hydrogen was connected to the flask and the reaction was stirred for 24 h. The reaction was diluted with ethanol, silica gel was added to the mixture and the solvent was removed under reduced pressure. The dry residue was purified through a short chromatographic column using 8% MeOH in CH₂Cl₂. The residue was purified by flash chromatography (5% MeOH in CH₂Cl₂) to give compound 6.23 as a white foam (33.7 mg, 70% over 2 steps); ¹H NMR (400 MHz, CDCl₃) δ 9.86-9.52 (m, 3H), 7.69-7.59 (m, 4H), 7.48-7.33 (m, 8H), 7.02 (s, 1H), 6.39 (dd, J=9.0, 5.3 Hz, 1H), 6.17 (t, J=6.7 Hz, 2H), 5.23-5.12 (m, 2H), 4.40 (dt, J=7.5, 3.8 Hz, 1H), 4.35-4.20 (m, 3H), 4.20-4.09 (m, 3H), 4.02 (q, J=4.0 Hz, 1H), 3.97 (dd, J=11.4, 2.7 Hz, 1H), 3.87 (dd, J=11.6, 2.5 Hz, 1H), 2.60-2.46 (m, 3H), 2.36-2.16 (m, 3H), 2.07-1.71 (m, 13H), 1.25 (s, 1H), 1.08 (s, 9H), 0.88 (s, 9H), 0.08 (s, 6H); ³¹P NMR (162 MHz, CDCl₃) δ 33.48, 33.17; HRMS (ESI) calc'd for C₅₆H₇₉N₆O₁₇P₂Si₂ [M+H]⁺ m/z=1225.451, found 1225.45396.

### Preparation of Final Compound 6.24

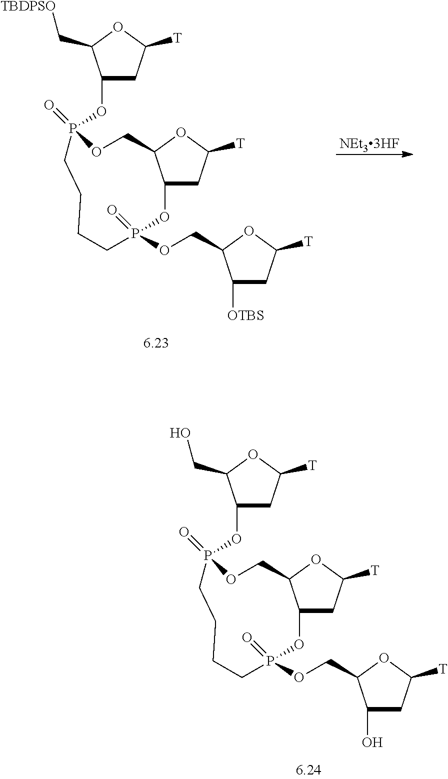

To a stirred solution of compound 6.23 (33 mg, 27 μmol) in THF (0.3 mL) was added NEt₃.3HF (27 μL, 165 mol). After stirring for 24 h at room temperature, the solvent was removed under reduced pressure and the residue was purified by flash chromatography (10% MeOH in CH₂Cl₂) to give compound 6.24 as a white foam (5 mg, 21%); ¹H NMR (300 MHz, Methanol-d₄) δ 7.81 (d, J=1.3 Hz, 1H), 7.57 (d, J=1.3 Hz, 1H), 7.43 (d, J=1.3 Hz, 1H), 6.34-6.24 (m, 2H), 6.14 (t, J=6.5 Hz, 1H), 5.33-5.13 (m, 2H), 4.50-4.15 (m, 9H), 4.12 (d, J=7.1 Hz, 1H), 4.10-4.03 (m, 1H), 3.82 (d, J=3.4 Hz, 2H), 2.79-2.38 (m, 4H), 2.36-2.05 (m, 5H), 1.91 (dd, J=5.2, 1.2 Hz, 16H); ³¹P NMR (162 MHz, CDCl₃) δ 34.15, 33.63.

### Example 2. Synthesis of Backbone Constrained Macrocycles, Compounds 6.52 and 6.54

### Preparation of Compound 6.44

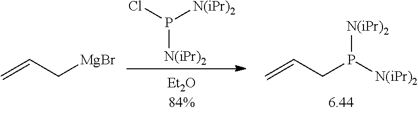

Bis(diisopropylamino)chlorophosphine (1.0 g, 3.56 mmol) was suspended in diethyl ether (12 mL), and was cooled to 0° C. in an ice bath with stirring. A 1 M commercial solution of allylmagnesium bromide (4 mL, 4 mmol) in ether was transferred to the chlorophosphine suspension. The reaction was allowed to come to room temperature and was stirred for one hour. The reaction mixture was filtered through a plug of celite, and the solids were rinsed with Et₂O. The filtrates were pooled, and the solvent was removed under reduced pressure. The residue was suspended in dry MeCN (10 ml). The reaction mixture was transferred to a separatory funnel and was extracted with hexanes (30 mL). The hexanes layer was washed with MeCN (2×15 ml). The hexanes layer was collected and passed thru a plug of cotton to remove particulates and traces of water. The filtrate was concentrated under reduced pressure to give 6.44 (815 mg, 84%). ¹H NMR (300 MHz, CDCl₃) δ 5.94-5.68 (m, 1H), 5.11-4.92 (m, 2H), 3.39 (dhept, J=10.5, 6.7 Hz, 4H), 2.53 (ddt, J=7.5, 2.9, 1.3 Hz, 2H), 1.19 (d, J=6.7 Hz, 12H), 1.09 (d, J=6.6 Hz, 12H); ¹³C NMR (75 MHz, CDCl₃) δ 135.82, 135.58, 115.56, 115.40, 46.63, 46.50, 35.23, 35.13, 24.51, 24.42, 24.20, 24.16, 24.12; ³¹P NMR (121 MHz, CDCl₃) δ 48.75.

### Preparation of Compound 6.45

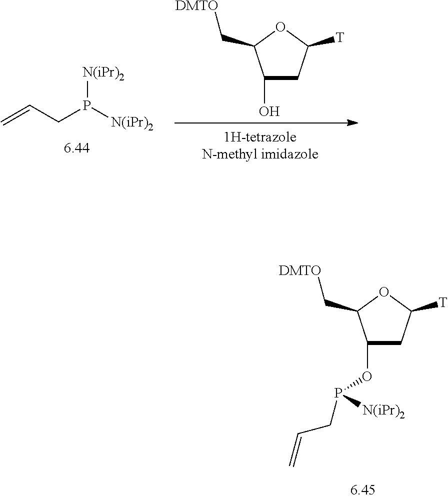

Commercially available 5′-ODMT thymidine (100 mg, 0.186 mmol) was coevaporated with toluene (×2) until complete dryness, then the flask was connected to the high vacuum pump for 20 min. Dry 5′-ODMT thymidine was dissolved in dry DMF (1.9 mL). In a second flask, a solution of 4,5-dicyanoimidazole (20.8 mg, 0.176 mmol) in acetonitrile (1.6 mL) was combined with 1-methyl imidazole (7 μL, 88 μmol). In a third flask a solution of 6.44 (75 mg, 0.27 mmol) in DMF (0.3 mL) was prepared. The solution containing 4,5-dicyanoimidazole and 1-methyl imidazole was added dropwise (via cannula) to the solution of 5′-ODMT thymidine followed by the dropwise addition of the solution of compound 6.44 (via cannula). Each flask was rinsed with a minimum amount of the corresponding solvent. The reaction was allowed to stir at room temperature for approx. 24 h then the solvent was removed under reduced pressure (keeping temp under 35° C.). The residue was purified by flash column chromatography (3:7 EtOAc/hex+1% NEt₃) to give compound 6.45 as a mixture of P-diastereoisomers (112 mg, 85%). ¹H NMR (400 MHz, CDCl₃) δ 8.18 (s, 1H), 7.63 (d, J=16.4 Hz, 1H), 7.43-7.32 (m, 2H), 7.33-7.18 (m, 8H), 6.86-6.74 (m, 4H), 6.38 (dt, J=8.3, 6.1 Hz, 1H), 5.80-5.55 (m, 1H), 5.17-4.92 (m, 2H), 4.53 (dd, J=11.3, 5.9 Hz, 1H), 3.79 (s, 6H), 3.63-3.38 (m, 3H), 3.31 (ddd, J=17.8, 10.5, 2.7 Hz, 1H), 2.59-2.36 (m, 2H), 2.27 (td, J=13.1, 12.5, 5.8 Hz, 2H), 1.40 (s, 3H), 1.19-0.97 (m, 12H); ¹³C NMR (101 MHz, CDCl₃) δ 164.28, 158.65, 150.69, 144.33, 144.21, 135.63, 135.46, 135.36, 135.31, 135.19, 132.53, 132.41, 132.28, 130.09, 130.04, 128.13, 128.09, 127.90, 127.06, 127.03, 117.50, 117.38, 117.17, 117.06, 111.22, 111.12, 86.87, 85.87, 84.90, 84.61, 63.70, 63.58, 60.30, 44.43, 44.34, 43.03, 40.33, 40.10, 37.93, 37.82, 24.53, 24.09, 20.95, 14.15, 11.66; ³¹P NMR (162 MHz, CDCl₃) δ 124.71, 124.27; HRMS (ESI) calc'd for C₄₀H₅₁N₃O₇P [M+H]⁺ m/z=716.3459, found 716.3452;

### Preparation of Compounds 6.46 and 6.47

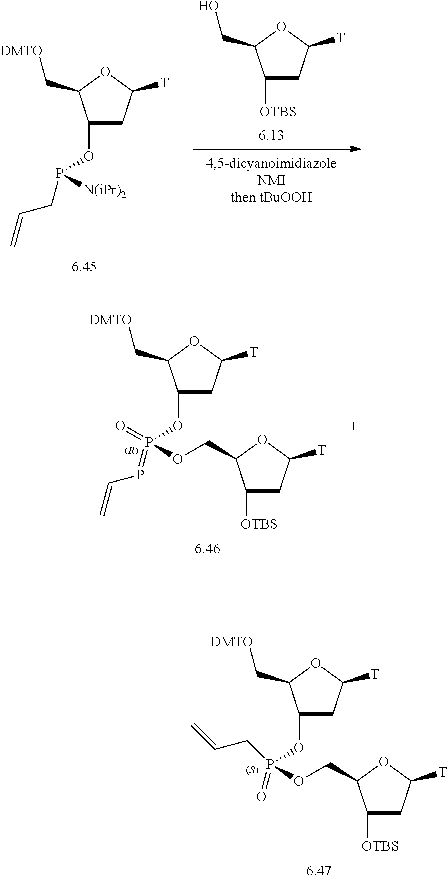

Previous to the reaction, nucleoside 6.13 was coevaporated with toluene to dryness (×2) and connected to the high vacuum pump for 15 min. Nucleoside 6.13 (404 mg, 1.13 mmol) was dissolved in anhydrous acetonitrile (12 mL). A solution of phosphoramidite 6.45 (1.0749 g, 1.50 mmol) in acetonitrile (15 mL) was added dropwise via cannula, followed by addition of a solution containing 4,5-dicyanoimidazole (199 mg, 1.68 mmol) and N-methylimidazole (13.4 μL, 0.168 mmol) in acetonitrile (17 mL) dropwise via cannula. The progress of the reaction was followed by TLC analysis (˜1 h) and MS. Upon completion, 5M tBuOOH in decanes (0.67 mL, 3.37 mmol) was added dropwise and the reaction mixture was stirred for 45 min. The reaction mixture was then diluted with EtOAc, cooled down to 0° C. and washed with a 10% NaHSO_{3(aq)} solution (1 mL) and a saturated solution of NaHCO₃ (5 mL). The aqueous solution was extracted with EtOAc and the combined organic layers were dried over Na₂SO₄, filtered, and concentrated under reduced pressure. The residue was adsorbed in silica and purified by flash chromatography using a Teledyne combiflash system (120 g cartridge) using a gradient hexanes/CH₂Cl₂ 7:2 with an increasing percentage of EtOH from 0% to 10%. Nucleoside 6.46 (308 mg, 27%) and 6.47 (281 mg, 25%) were obtained as white foams. 6.46: ¹H NMR (300 MHz, CDCl₃) δ 10.26-9.93 (m, 2H), 7.51 (s, 1H), 7.43-7.29 (m, 3H), 7.26-7.11 (m, 7H), 6.80 (d, J=8.5 Hz, 4H), 6.41 (dd, J=8.4, 5.5 Hz, 1H), 6.26-6.10 (m, 1H), 5.95-5.51 (m, 1H), 5.41-5.01 (m, 3H), 4.65-3.83 (m, 5H), 3.74 (s, 6H), 3.48 (d, J=10.7 Hz, 1H), 3.31 (d, J=9.0 Hz, 1H), 2.71-2.49 (m, 3H), 2.48-2.31 (m, 1H), 2.30-2.17 (m, 1H), 2.11 (dt, J=13.3, 6.7 Hz, 1H), 1.85 (s, 3H), 1.36 (s, 3H), 0.85 (s, 9H), 0.05 (s, 6H). ¹³C NMR (75 MHz, CDCl₃) δ 164.20, 164.07, 158.70, 150.74, 150.49, 143.93, 135.71, 135.00, 134.93, 131.72, 130.02, 128.07, 127.95, 127.18, 126.27, 126.11, 121.14, 120.95, 119.05, 113.25, 111.65, 111.06, 110.77, 87.13, 85.48, 85.04, 84.95, 84.55, 84.14, 71.45, 68.72, 64.96, 63.17, 55.16, 40.40, 39.46, 32.68, 30.85, 25.66, 25.62, 17.82, 12.41, 12.32, 11.63, −4.71, −4.77, −4.92; ³¹P NMR (162 MHz, CDCl₃) δ 27.84; HRMS (ESI) calc'd for C₅₀H₆₄N₄O₁₃PSi [M+H]⁺ m/z=1009.3791, found 1009.3796. 6.47: ¹H NMR (300 MHz, CDCl₃) δ 10.18 (d, J=38.1 Hz, 2H), 7.63 (s, 2H), 7.36 (dd, J=8.5, 6.9 Hz, 2H), 7.27-7.17 (m, 7H), 6.85-6.79 (m, 5H), 6.42-6.33 (m, 1H), 6.21-6.07 (m, 1H), 5.86-5.60 (m, 1H), 5.33-5.19 (m, 2H), 4.42-3.85 (m, 5H), 3.77 (s, 6H), 3.56-3.31 (m, 2H), 2.74 (dd, J=22.0, 7.3 Hz, 2H), 2.64-2.08 (m, 4H), 1.87 (s, 3H), 1.41-1.33 (s, 3H), 0.86 (s, 9H), 0.06 (s, 6H). ¹³C NMR (75 MHz, CDCl₃) δ 164.77, 164.69, 158.88, 158.78, 151.13, 150.91, 150.47, 144.33, 144.06, 140.33, 136.50, 135.86, 135.40, 135.32, 135.07, 134.98, 131.12, 130.15, 129.36, 129.22, 128.17, 127.40, 127.25, 126.02, 125.87, 121.79, 121.60, 116.07, 113.98, 113.41, 113.36, 113.22, 111.79, 111.50, 111.14, 110.49, 87.37, 87.07, 85.96, 84.90, 84.53, 72.64, 71.36, 63.74, 63.19, 55.35, 32.73, 25.71, 17.93, 12.60, 11.73, −4.60, −4.82; ³¹P NMR (162 MHz, CDCl₃) δ 28.56; HRMS (ESI) calc'd for C₅₀H₆₄N₄O₁₃PSi [M+H]⁺ m/z=1009.3791, found 1009.3801.

### Preparation of Compound 6.48

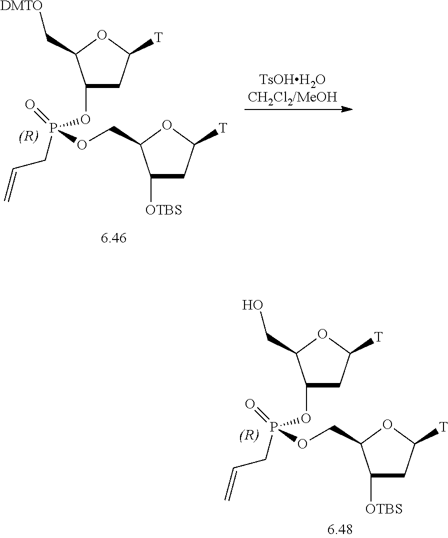

To a stirred solution of 6.46 (72 mg, 0.73 mmol) in 3:1 CH₂Cl₂/MeOH (7.3 mL) was added p-toluenesulfonic acid monohydrate (16.6 mg, 0.876 mmol) at 0° C. for 1 h. Once the reaction was completed, solid Na₂CO₃ was added and the mixture was stirred until the orange color disappeared. Water (2 mL) was added and the resulting mixture was extracted with CH₂Cl₂ (4×3 mL). The organic extractions were combined, dried over Na₂SO₄ and concentrated under reduced pressure. The residue was purified by flash chromatography (0% to 5% MeOH in CH₂Cl₂) to give nucleoside 6.48 (42.2 mg, 83%); ¹H NMR (400 MHz, CDCl₃) δ 10.18 (s, 1H), 9.98 (s, 1H), 7.56 (s, 1H), 7.39 (s, 1H), 6.26-6.16 (m, 2H), 5.74 (ddq, J=17.1, 10.0, 7.2 Hz, 1H), 5.28-5.15 (m, 3H), 4.39-4.28 (m, 1H), 4.24-4.08 (m, 3H), 4.00-3.93 (m, 2H), 3.76 (q, J=15.2, 14.2 Hz, 2H), 2.68 (dd, J=22.2, 7.4 Hz, 2H), 2.52-2.38 (m, 1H), 2.37-2.12 (m, 3H), 1.88 (s, 3H), 1.82 (s, 3H), 0.85 (s, 9H), 0.04 (s, 6H); ³¹P NMR (162 MHz, CDCl₃) δ 28.21; ¹³C NMR (101 MHz, CDCl₃) δ 164.65, 164.46, 150.74, 136.58, 136.31, 126.35, 126.24, 121.35, 121.20, 111.45, 111.14, 86.11, 85.60, 85.12, 71.49, 65.79, 61.92, 40.19, 39.07, 32.56, 31.17, 25.73, 17.94, 12.52, 12.39, −4.61, −4.82; ³¹P NMR (162 MHz, CDCl₃) δ 28.25; HRMS (ESI) calc'd for C₂₉H₄₆N₄O₁₁PSi [M+H]⁺ m/z=685.2664, found 685.2697.

### Preparation of Compound 6.49

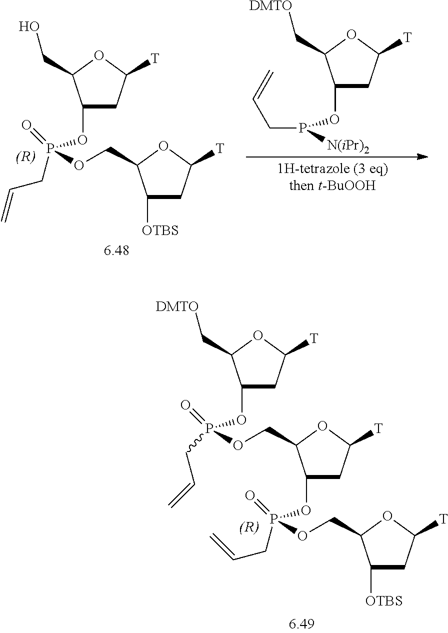

To a stirred solution of 6.48 (40 mg, 0.0584 mmol) in MeCN (0.7 mL) was added sequentially a solution of phosphoramidite 6.45 (137.8 mg, 0.192 mmol) in MeCN (2 mL), a 0.45 M solution of 1H-tetrazole (0.2 mL, 0.09 mmol) and N-methylimidazole (5 μL, 0.062 mmol). The reaction mixture was stirred for 2 h, then a 5 M solution of tBuOOH in decanes (0.07 mL, 0.35 mmol) was added dropwise and stirred for additional 30 min. The reaction mixture was diluted with EtOAc, cooled down to 0° C. and combined with a sat solution of NaHCO₃ (2 mL) and a 10% solution of NaHSO₃ (1 mL). The biphasic mixture was stirred for 5 min, then the layers were separated, and the aqueous layer was extracted with EtOAc. The organic combined were dried over Na₂SO₄ and concentrated under reduced pressure. The residue was purified by flash chromatography (0% to 6% MeOH in CH₂Cl₂) to give nucleoside 6.49 as an inseparable mixture of isomers (75 mg, 91%). ³¹P NMR (162 MHz, CDCl₃) δ 28.62, 28.18, 28.13, 28.00; HRMS (ESI) calc'd for C₆₃H₈₀N₆O₁₉P₂Si [M+Na]⁺ m/z=1337.4615, found 1337.4635.

### Preparation of Compounds 6.50 and 6.51

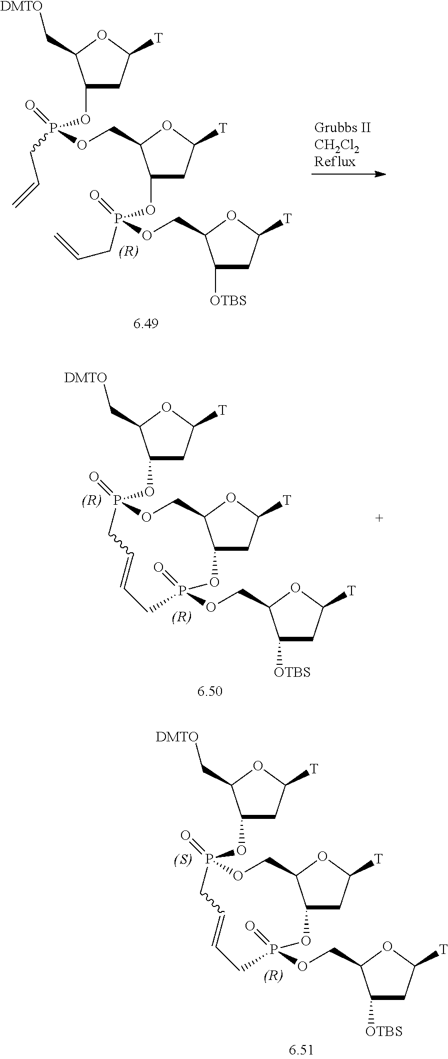

To a stirred solution of 6.49 (32 mg, 24.3 mol) in CH₂Cl₂ (1 mL) was added second generation Grubbs catalyst (2 mg, 2.3 μmol) in a sealed tube. The reaction mixture was heated at 45° C. for 24 h, then the solvent was removed under reduced pressure and the crude residue was purified by flash chromatography (4% to 8% iPrOH in CH₂Cl₂) to give 6.50 (6.7 mg, 32%) and 6.51 (6.3 mg, 31%). Both isolated products contained an inseparable mixture of cis trans isomers. Compound 6.50: ³¹P NMR (202 MHz, CDCl₃) δ 28.06, 27.94, 27.81, 27.69. Compound 6.51: ³¹P NMR (202 MHz, CDCl₃) δ 27.16, 27.03, 26.67, 26.54.

### Preparation of Compound 6.52

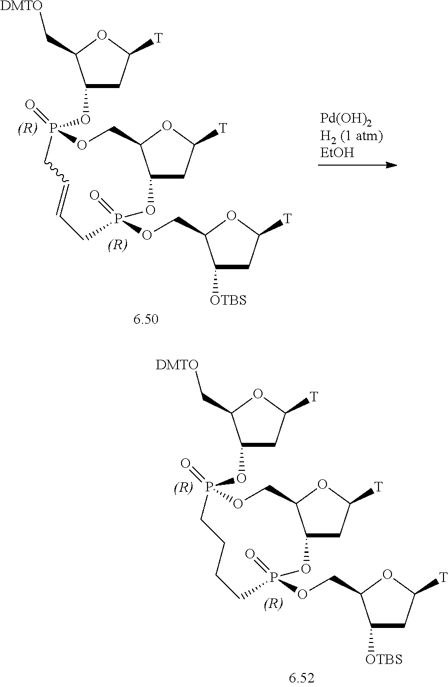

To a stirred solution of 6.50 (6 mg, 4.6 μmol) in ethanol (0.5 mL) was added 20% Pd(OH)₂ (˜2 mg). The flask was degassed under vacuum and refilled with hydrogen (×3). A balloon filled with hydrogen was connected to the flask and the reaction was stirred for 24 h. Once all the starting material was consumed, the reaction mixture was diluted with ethanol and filtrated through a celite pad rinsing with more ethanol. The resulting solution was purified by flash chromatography (0% to 9% MeOH in CH₂Cl₂) to give nucleoside 6.52 (2.5 mg, 54%); ¹H NMR (400 MHz, Methanol-d₄) δ 7.80 (s, 1H), 7.54 (s, 1H), 7.41 (s, 1H), 6.37-6.29 (m, 1H), 6.22 (t, J=6.8 Hz, 1H), 6.09 (t, J=6.7 Hz, 1H), 5.31 (dd, J=8.1, 4.1 Hz, 1H), 5.13 (s, 1H), 4.57-4.49 (m, 2H), 4.37-4.08 (m, 2H), 4.08-4.01 (m, 1H), 3.98-3.90 (m, 1H), 3.83-3.75 (m, 2H), 2.74-2.66 (m, 1H), 2.59-2.51 (m, 1H), 2.61-2.36 (m, 3H), 2.30-2.25 (m, 1H), 2.20-2.09 (m, 2H), 2.09-1.97 (m, 2H), 1.96-1.75 (m, 11H), 1.16 (d, J=6.1 Hz, 4H), 0.94 (s, 9H), 0.22-0.01 (s, 6H); 31P NMR (162 MHz, MeOD) δ 33.92, 33.50; HRMS (ESI) calc'd for C₄₀H₆₁N₆O₁₇P₂Si [M+H]⁺ m/z=987.3332, found 987.3343.

### Preparation of Compound 6.54

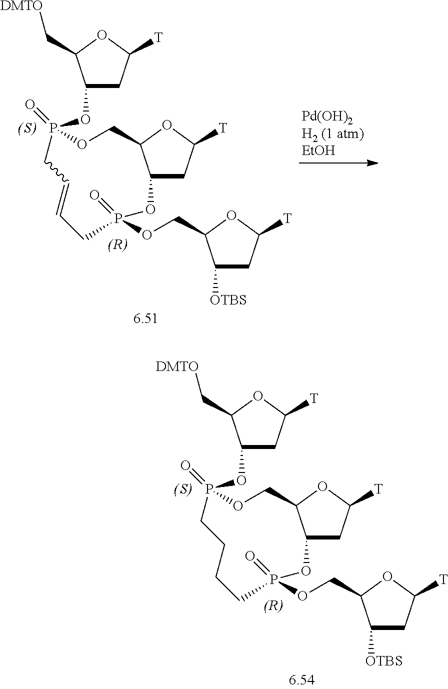

To a stirred solution of 6.51 (6 mg, 4.6 μmol) in ethanol (0.5 mL) was added 20% Pd(OH)₂ (˜2 mg). The flask was degassed under vacuum and refilled with hydrogen (×3). A balloon filled with hydrogen was connected to the flask and the reaction was stirred for 24 h. Once all the starting material was consumed, the reaction mixture was diluted with ethanol and filtrated through a celite pad rinsing with more ethanol. The resulting solution was purified by flash chromatography (0% to 9% MeOH in CH₂Cl₂) to give nucleoside 6.54 (2.4 mg, 52%); ¹H NMR (400 MHz, Methanol-d₄) δ 7.80 (s, 1H), 7.54 (s, 1H), 7.42 (s, 1H), 6.33-6.28 (m, 1H), 6.23 (t, J=6.8 Hz, 1H), 6.16-6.10 (m, 1H), 5.30-5.21 (m, 1H), 5.19-5.11 (m, 1H), 4.57-4.50 (m, 1H), 4.41-4.14 (m, 3H), 4.11-3.77 (m, 4H), 2.76-2.66 (m, 2H), 2.65-2.40 (m, 4H), 2.27 (t, J=5.7 Hz, 2H), 2.19-2.00 (m, 5H), 1.95-1.79 (m, 11H), 1.00 (d, J=6.7 Hz, 4H), 0.93 (s, 9H), 0.14 (s, 3H); 31P NMR (162 MHz, MeOD) δ 34.02, 33.38; HRMS (ESI) calc'd for C₄₀H₆₁N₆O₁₇P₂Si [M+H]⁺ m/z=987.3332, found 987.3326.

### Example 3. Synthesis of Oligomeric Compounds Comprising Oxygen-Containing Constrained Backbone Nucleic Acids, Compounds 6.80a and 6.80b

Ring size and position of the heteroatom can be varied by changing the starting materials in the first step of the synthesis.

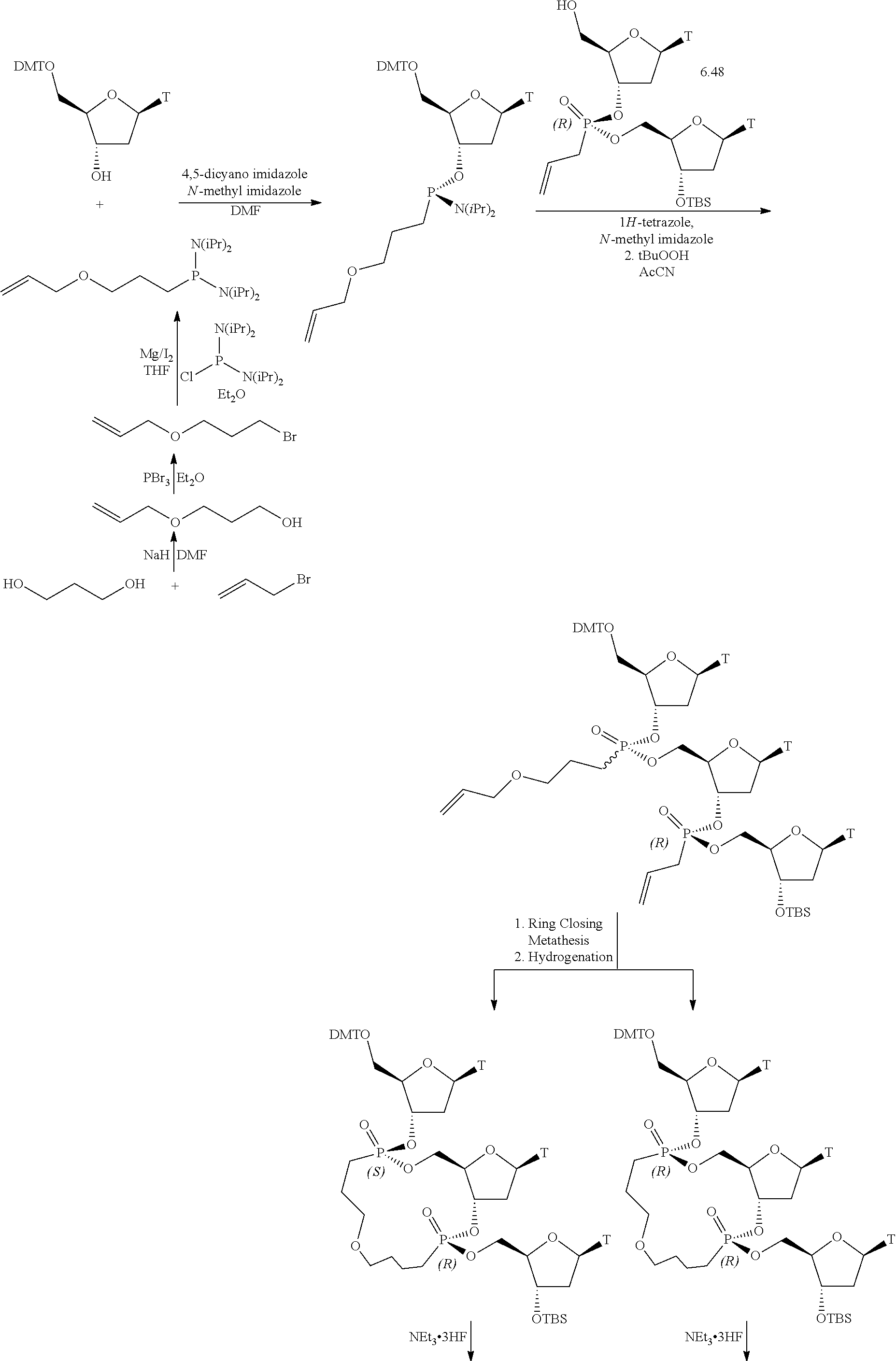

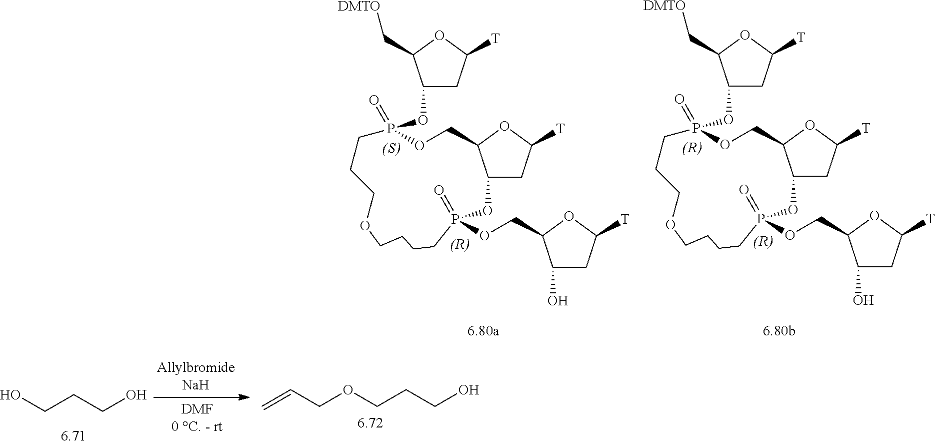

### Preparation of 3-(allyloxy)propan-1-ol 6.72

Propane 1,3-diol 6.71 (10 g, 0.132 mol) was dissolved in dry DMF (100 ml) under argon and cooled to 0° C. NaH (5 g, 0.131 mol, 60% in mineral oil) was added portion wise for 30 mins and the resulting suspension was stirred further for 15 mins. Allyl bromide (11.4 ml, 0.131 mol) was added dropwise over 10 mins. Reaction mixture was slowly allowed to rt and stirred overnight. The reaction mixture was poured into ice cold water and aqueous layer was extracted with ether (2×100 ml). The combined organic layer was washed with cold water (2×50 ml), dried over Na₂SO₄, filtered and concentrated under reduced pressure. The crude mass was purified by flash column chromatography on silica gel (gradient from 100% hexanes to hexanes/ethyl acetate 8:2). 3-(allyloxy)propan-1-ol 6.72 (8.6 g, 57% yield) was obtained as pale yellow liquid.

¹H NMR (400 MHz, CDCl₃) δ 5.84 (ddt, J=21.7, 10.6, 5.4 Hz, 1H), 5.29-5.05 (m, 2H), 3.93 (dt, J=5.7, 1.5 Hz, 2H), 3.69 (t, J=5.8 Hz, 2H), 3.55 (t, J=5.9 Hz, 2H), 2.80 (s, 1H), 1.78 (p, J=5.9 Hz, 2H).

¹³C NMR (101 MHz, CDCl₃) δ 134.6, 117.0, 72.0, 68.98, 61.3, 32.2.

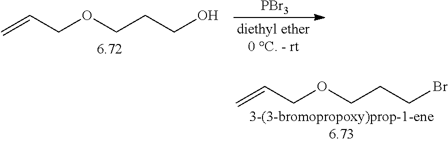

### Preparation of 3-(3-bromopropoxy)prop-1-ene 6.73

To a solution of 3-(allyloxy)propan-1-ol 6.72 (5 g, 43.1 mmol) in anhydrous diethylether at 0° C. phosphorus tribromide (4.0 mL, 43.1 mmol) was added dropwise under argon and resulting mixture was slowly allowed to reflux. The progress of the reaction was monitored by TLC. After complete consumption of alcohol (approx. 30 min) the reaction mixture was poured onto crushed ice and extracted with ether. The combined organic layers were washed with cold water (2×50 ml), dried over Na₂SO₄ and concentrated under reduced pressure. The crude mass was purified by flash column chromatography on silica gel (gradient from 100% hexanes to hexanes/ethyl acetate 9:1). 3-(3-bromopropoxy)prop-1-ene 6.73 (4.32 g, 56% yield) was obtained as pale yellow liquid.

¹H NMR (400 MHz, CDCl₃) δ 5.90 (ddt, J=17.3, 10.4, 5.6 Hz, 1H), 5.31-5.13 (m, 2H), 3.97 (dt, J=5.6, 1.5 Hz, 2H), 3.53 (dt, J=16.5, 6.2 Hz, 4H), 2.10 (p, J=6.3 Hz, 2H).

¹³C NMR (101 MHz, CDCl₃) δ 134.8, 117.1, 72.1, 67.7, 33.0, 30.8.

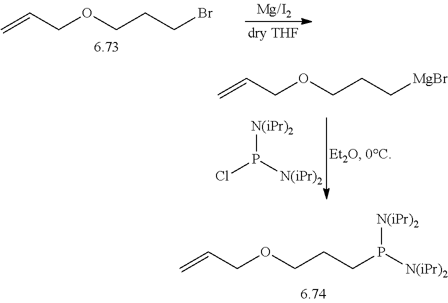

### Preparation of Phosphine 6.74

To a suspension of Mg (340 mg, 13.98 mmol) and pinch of I₂ in dry THF (23 ml) was added dropwise bromocompound 6.73 (1 g, 5.58 mmol) with stirring at room temperature. In a separate oven dried flask, bis(diisopropylamino)chlorophosphine (1.5 g, 5.62 mmol) was suspended in 50 ml dry Et₂O under argon atm and cooled to 0° C. with constant stirring. The above freshly prepared solution of Grignard reagent (10 mL, 0.56 M in THF) was cannulated into the stirred solution of chlorophosphine suspension dropwise and the reaction mixture was allowed to stir at 0° C. for 1 h. The progress of the reaction was monitored by ³¹P NMR (CDCl₃, 400 MHz, 64 scans). ³¹P NMR showed major peaks at 47.35 ppm (product) and 20.6 ppm (minor). The reaction mixture was filtered through a plug of celite and the solids were rinsed with Et₂O. The solvent was removed in vacuo. The residue was suspended in dry MeCN, transferred to a separatory funnel and extracted with hexanes (2×100 mL). The hexanes layer was washed with MeCN (25 ml). The hexanes layer was collected and passed through a plug of cotton to remove particulates and traces of water. The filtrate was concentrated at 30° C. and dried under high vacuum briefly. Used immediately in next step without further purification. 1.6 g (86% yield)

¹H NMR (400 MHz, CDCl₃) δ 5.98-5.81 (m, 1H), 5.29-5.11 (m, 2H), 3.96 (dt, J=5.6, 1.5 Hz, 2H), 3.49 (td, J=6.6, 3.7 Hz, 2H), 3.37 (m, 4H), 1.65 (m, 4H), 1.20-1.12 (m, 12H), 1.07 (d, J=6.7 Hz, 12H).

³¹P NMR (162 MHz, CDCl₃) δ 47.35.

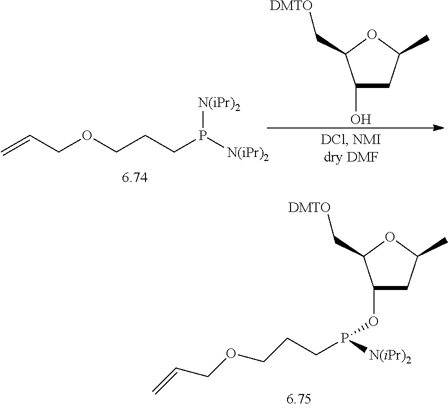

### Preparation of Phosphoramidite 6.75

5′-ODMT thymidine (2.0 g, 3.67 mmol, 1 equiv.) was co-evaporated with toluene (2×10 mL) until complete dryness. The flask was connected to the high vacuum pump for at least 30 min. The so dried 5′-ODMT thymidine was dissolved in dry DMF (10 mL) under argon. 4,5-dicyanoimidazole (381 mg, 3.28 mmol, 0.88 equiv.) and 1-methyl imidazole (132 mg, 1.6 mmol, 0.44 equiv.) were added under argon. A solution of phosphine 6.74 (1.5 g, 4.53 mmol, 1.23 equiv.) in dry DMF (5 mL) was added dropwise at rt. The reaction mixture was allowed to stir at room temperature for 12 h until complete consumption of the 5′-ODMT thymidine (monitored by TLC analysis). The volatiles were removed under reduced pressure (keeping the temperature of the rotavapor water bath under 40° C.). The resulting oil was dissolved in a minimum amount of dichloromethane and immediately purified by flash silica gel column chromatography (gradient, from 100% Hexanes+1% Et3N to 3:7 EtOAc/hexanes+1% Et3N) to give the desired phosphoramidite 6.75 as a white solid (2.2 g, 77% yield) as a mixture of P-diastereoisomers.

¹H NMR (400 MHz, CDCl₃) δ 7.68 (d, J=1.4 Hz, 1H), 7.40 (dd, J=8.3, 1.3 Hz, 2H), 7.36-7.19 (m, 8H), 6.90-6.77 (m, 4H), 6.40 (m, 1H), 6.01-5.85 (m, 1H), 5.34-5.13 (m, 2H), 4.57 (m, 1H), 4.18-3.93 (m, 4H), 3.81 (s, 6H), 3.56-3.26 (m, 4H), 2.53-2.23 (m, 2H), 1.76-1.52 (m, 4H), 1.40 (t, J=1.5 Hz, 3H), 1.18 (d, J=6.6 Hz, 3H), 1.07 (m, 9H).

³¹P NMR (162 MHz, CDCl₃) δ 126.8, 126.5.

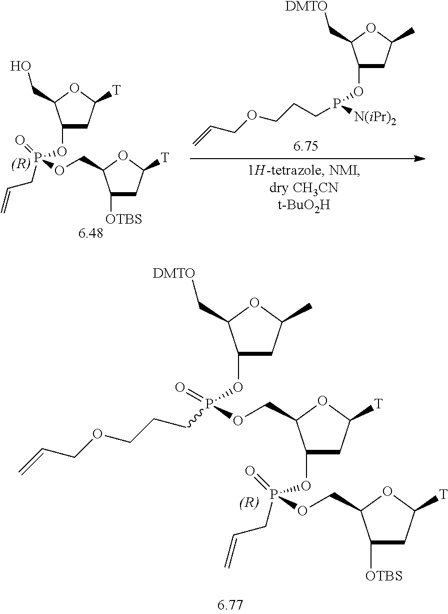

### Preparation of Nucleotide Trimer 6.77

To a stirred solution of dimer 6.48 (400 mg, 0.584 mmol, 1 equiv.) in anhydrous acetonitrile (10 mL) under argon were added sequentially a 3% solution of 1H-tetrazole (2.2 mL, 0.93 mmol, 1.6 equiv.) in MeCN and N-methylimidazole (42 mg, 0.512 mmol, 0.88 equiv.) were added. A solution of phosphoramidite (1.36 g, 1.76 mmol, 3 equiv.) in anhydrous acetonitrile (10 mL) was added dropwise. The reaction mixture was stirred for 4 h then a 5 M solution of tert-butyl hydroperoxide in decanes (0.7 mL, 3.50 mmol, 6 equiv.) was added dropwise and the mixture was stirred for another 30 min. The reaction mixture was diluted with ethyl acetate (50 mL), aqueous solution of 10% NaHCO₃ (2 mL) and 10% NaHSO₃ (2 mL) was added. The biphasic mixture was stirred for 10 min, then the layers were separated, and the aqueous layer was extracted with ethyl acetate. The combined organic layers were dried over dried Na₂SO₄ and concentrated under reduced pressure. The residue was purified by flash column chromatography on silica gel (gradient from 100% dichloromethane to dichloromethane/methanol 96:4) to give nucleoside trimer 6.77 as mixture of P-diastereoisomers (580 mg, 73% yield).

¹H NMR (400 MHz, CDCl₃) δ 9.54 (s, 1H), 9.30 (s, 1H), 9.20 (s, 1H), 7.69-7.13 (m, 14H), 6.85 (dd, J=9.1, 2.8 Hz, 4H), 6.48-6.28 (m, 1H), 6.20 (q, J=6.3 Hz, 1H), 5.99-5.67 (m, 2H), 5.46-4.99 (m, 6H), 4.53-4.08 (m, 7H), 4.08-3.89 (m, 3H), 3.81 (d, J=2.4 Hz, 6H), 3.54-3.26 (m, 4H), 2.69 (m, 2H), 2.58-2.36 (m, 2H), 2.30 (m, 3H), 2.01-1.65 (m, 13H), 0.91 (s, 9H), 0.10 (d, J=2.7 Hz, 6H).

³¹P NMR (162 MHz, CDCl₃) δ 34.2, 33.8, 28.3, 28.2.

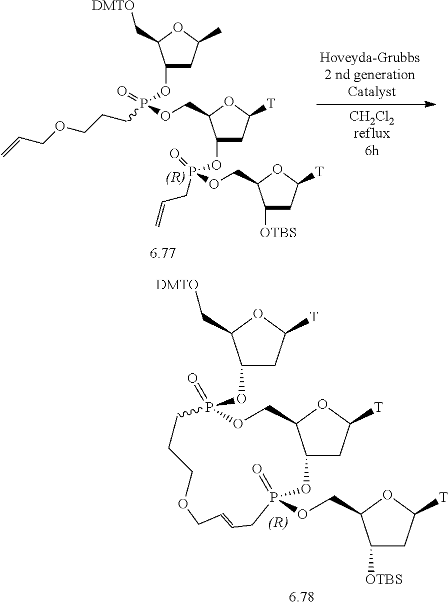

Preparation of Compound 6.78 from Ring Closing Metathesis of Nucleotide Trimer 6.77

The nucleoside trimer 6.77 (500 mg, 0.36 mmol) was dissolved in degassed (by bubbling argon for 30 min) anhydrous dichloromethane (200 mL) under argon. Second generation Hoveyda-Grubbs catalyst (22 mg, 0.036 mmol) was added to the phosphonates solution and the reaction mixture was heated to reflux for 6 h (the progress of the reaction was followed by TLC and MS spectrometry). The reaction mixture was concentrated under vacuum and the crude mass was pre-purified from the catalyst by flash column chromatography using a short pad of silica gel (isocratic 100% ethyl acetate, then DCM/methanol 90:10). The collected fractions containing the products were concentrated and subjected to purification by flash column chromatography (gradient from 100% dichloromethane to dichloromethane/methanol 95:5). The volatiles were removed under reduced pressure and the mixture of desired macrocyclic nucleoside 6.78 (450 mg, 92% yield) were directly subjected to the following hydrogenation step.

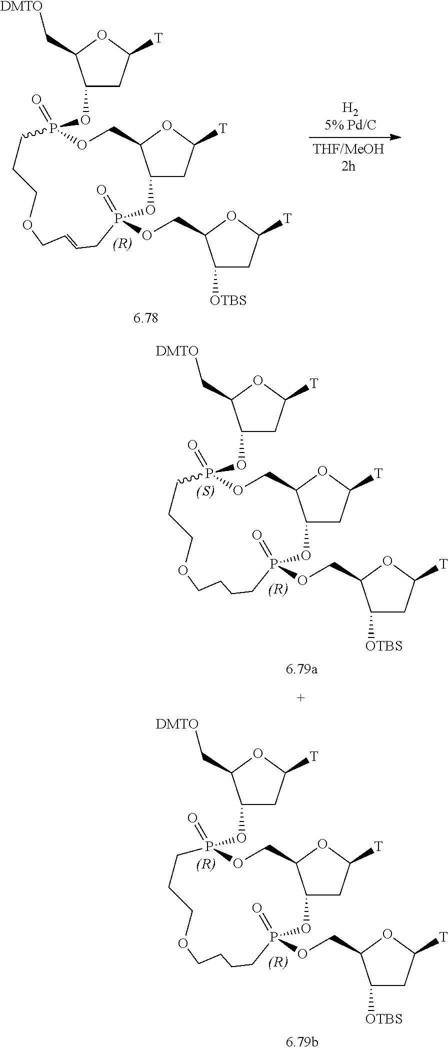

### Preparation of Compounds 6.79a and 6.79b

To a solution of macrocycles 6.78 (400 mg, 0.297 mmol) in a 1:1 mixture of MeOH/THF (10 mL), 5% Pd/C pre-treated with 2,6-lutidine (126 mg, 0.059 mmol) was added. The system was flushed with argon, and then stirred under hydrogen atmosphere for 2 h (the progress of the reaction was monitored by TLC and mass spectrometry). After completion, the reaction mixture was diluted with DCM and filtered through a PTFE 0.45 m, 25 mm syringe filter (Fischer Scientific). The filter was rinsed with dichloromethane. The filtrate was concentrated under reduced pressure to obtain the desired product as a mixture of macrocycle 6.79a and 6.79b (360 mg, 90% yield).

The separation of the two diastereoisomers was performed by preparative silica TLC (1000 m thickness, 20×20 cm, 8% isopropanol in dichloromethane as system solvent) to give pure macrocycle 6.79a (180 mg, 45%) and pure macrocycle 6.79b (140 mg, 35%).

Compound 6.79a

¹H NMR (400 MHz, CDCl₃) δ 9.45 (s, 2H), 9.39 (s, 1H), 7.50 (d, J=1.5 Hz, 1H), 7.42 (d, J=1.4 Hz, 1H), 7.38-7.31 (m, 2H), 7.31-7.18 (m, 7H), 6.88 (d, J=1.4 Hz, 1H), 6.85-6.78 (m, 4H), 6.41 (dd, J=8.8, 5.5 Hz, 1H), 6.21 (t, J=6.8 Hz, 1H), 5.90 (t, J=6.5 Hz, 1H), 5.25 (t, J=6.4 Hz, 1H), 5.12-5.04 (m, 1H), 4.37 (dt, J=6.9, 3.6 Hz, 1H), 4.30 (q, J=2.3 Hz, 1H), 4.18 (m, 4H), 4.00 (m, 2H), 3.81-3.73 (m, 6H), 3.59-3.34 (m, 6H), 2.61-2.38 (m, 4H), 2.29-2.08 (m, 3H), 1.91 (d, J=1.1 Hz, 3H), 2.04-1.69 (m, 10H), 1.76 (s, 3H), 1.35 (d, J=1.2 Hz, 3H), 0.88 (s, 9H), 0.86-0.77 (m, 1H), 0.07 (s, 6H).

¹³C NMR (101 MHz, CDCl₃) δ 164.2, 163.8, 163.8, 158.9, 158.8, 150.9, 150.6, 150.1, 144.3, 136.3, 136.2, 135.3, 135.2, 130.2, 130.2, 128.2, 128.2, 127.3, 113.5, 111.9, 111.3, 111.3, 87.3, 86.9, 85.9, 85.3, 85.3, 84.9, 84.4, 83.2, 77.1, 77.0, 75.1, 71.8, 69.9, 69.0, 64.7, 63.3, 55.4, 40.4, 39.4, 38.4, 29.7, 29.5, 26.2, 25.8, 24.8, 22.8, 22.2, 20.8, 20.1, 12.6, 12.5, 11.8, −4.5, −4.7.

³¹P NMR (162 MHz, CDCl₃) δ 33.86, 33.81.

Compound 6.79b

¹H NMR (400 MHz, Chloroform-d) δ 9.7 (s, 1H), 9.6 (s, 2H), 7.5 (d, J=1.4 Hz, 1H), 7.4-7.3 (m, 3H), 7.3-7.2 (m, 7H), 7.1 (d, J=1.6 Hz, 1H), 6.9-6.8 (m, 4H), 6.4 (dd, J=8.5, 5.5 Hz, 1H), 6.2-6.1 (m, 2H), 5.2 (t, J=6.2 Hz, 1H), 5.0 (m, 1H), 4.4-4.3 (m, 2H), 4.3-4.1 (m, 5H), 4.0 (m, 1H), 3.8 (s, 6H), 3.5-3.3 (m, 6H), 2.7-2.5 (m, 2H), 2.4 (m, 2H), 2.3-2.2 (m, 2H), 2.0-1.7 (m, 10H), 1.92 (d, J=1.1 Hz, 3H), 1.86 (d, J=1.1 Hz, 3H), 1.4 (d, J=1.1 Hz, 3H), 0.9 (s, 9H), 0.1 (s, 6H).

¹³C NMR (101 MHz, Chloroform-d) δ 164.3, 164.0, 164.0, 158.9, 150.8, 150.7, 150.4, 144.2, 136.4, 135.7, 135.3, 135.2, 135.2, 130.3, 130.2, 128.3, 128.2, 127.4, 113.5, 111.8, 111.7, 111.4, 87.3, 86.1, 85.7, 85.3, 85.3, 84.9, 84.4, 75.2, 71.7, 69.9, 69.0, 64.9, 64.8, 63.4, 55.4, 40.3, 39.5, 38.3, 29.8, 25.8, 24.9, 23.0, 12.6, 12.6, 11.9, −4.5, −4.7.

³¹P NMR (162 MHz, CDCl₃) δ 33.42, 33.25.

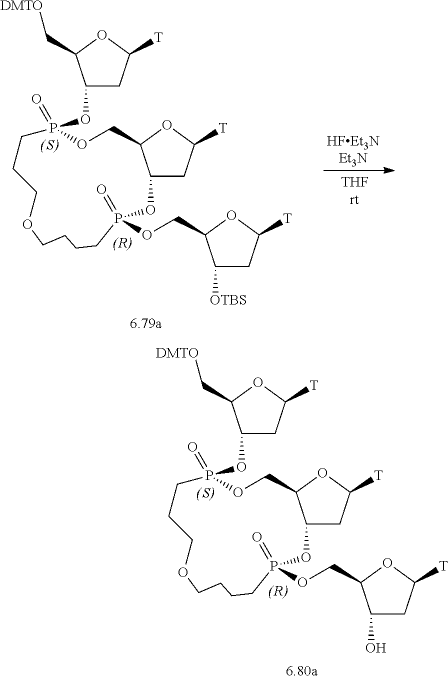

### Preparation of Macrocyclic Nucleotide 6.80a

To a stirred solution of macrocyclic nucleotide 6.79a (180 mg, 0.133 mmol, 1 equiv.) and anhydrous triethylamine (38 μL, 0.266 mmol, 2 equiv.) in anhydrous tetrahydrofuran (5 mL) under argon, triethylamine trihydrofluoride (65 μL, 0.399 mmol, 3 equiv.) was added. The progress of the reaction was followed by TLC analysis. Upon completion, the reaction mixture was diluted with ethyl acetate (100 mL), then a saturated solution of NaHCO₃ was added and the resulting mixture was stirred for 10 min. The layers were separated, and the aqueous layer was extracted with ethyl acetate (20 mL). The organic layers were combined, dried over Na₂SO₄, filtered and the solvent evaporated under reduced pressure. The crude was purified by flash column chromatography on silica gel (gradient from 100% dichloromethane to dichloromethane/methanol 93:7) to give compound 6.80a (130 mg, 79%)

¹H NMR (400 MHz, 95% CDCl₃+5% MeOH-d4) δ 7.5 (d, J=1.4 Hz, 1H), 7.3-7.3 (m, 4H), 7.3-7.1 (m, 7H), 6.9 (d, J=1.4 Hz, 1H), 6.7 (d, J=8.9 Hz, 4H), 6.3 (dd, J=8.9, 5.4 Hz, 1H), 6.1 (t, J=6.6 Hz, 1H), 5.9 (t, J=6.7 Hz, 1H), 5.2-5.1 (m, 1H), 5.03-4-94 (m, 1H), 4.35-4.28 (m, 1H), 4.3-4.1 (m, 5H), 4.0-3.8 (m, 2H), 3.7 (s, 6H), 3.46-3.39 (m, 3H), 3.4-3.3 (m, 4H), 2.56-2.44 (m, 2H), 2.4-2.2 (m, 3H), 2.2-2.0 (m, 2H), 1.93-1.61 (m, 8H), 1.84 (d, J=1.2 Hz, 3H), 1.73 (d, J=1.2 Hz, 3H), 1.6-1.5 (m, 1H), 1.3 (d, J=1.2 Hz, 3H), 1.2 (m, 1H), 0.8 (m, 1H).

¹³C NMR (101 MHz, 95% CDCl₃+5% MeOH-d4) δ 164.48, 164.36, 164.17, 158.75, 158.72, 150.81, 150.55, 150.35, 144.07, 136.10, 135.86, 135.37, 135.10, 135.06, 130.07, 130.03, 128.06, 128.03, 127.25, 113.32, 111.65, 111.35, 111.00, 87.26, 86.13, 85.62, 84.83, 84.80, 84.72, 84.66, 84.26, 82.90, 82.82, 82.75, 77.36, 75.45, 75.39, 70.27, 69.72, 68.82, 68.69, 64.85, 64.78, 64.05, 63.99, 63.20, 55.21, 39.76, 39.22, 39.17, 38.03, 29.39, 29.23, 25.83, 24.45, 22.60, 22.55, 22.30, 20.91, 19.78, 19.73, 12.27, 12.21, 11.51.

³¹P NMR (162 MHz, 95% CDCl₃+5% MeOH-d4) δ 34.66, 34.21.

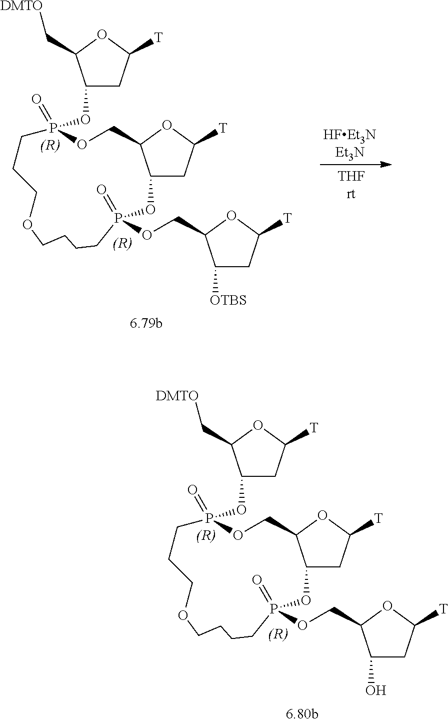

### Preparation of Macrocyclic Nucleotide 6.80b

To a stirred solution of macrocycle 6.79b (140 mg, 0.103 mmol, 1 equiv.) and anhydrous triethylamine (30 μL, 0.206 mmol, 2 equiv.) in anhydrous tetrahydrofuran (4 mL), under argon, triethylamine trihydrofluoride (50 L, 0.309 mmol, 3 equiv.) was added. The progress of the reaction was followed by TLC analysis. Upon completion, the reaction mixture was diluted with ethyl acetate (100 mL), then a saturated solution of NaHCO₃ was added and the resulting mixture was stirred for 10 min. The layers were separated, and the aqueous layer was extracted with ethyl acetate (20 mL). The organic layers were combined, dried over Na2SO4, filtered and the solvent evaporated under reduced pressure. The crude was purified by flash column chromatography on silica gel (gradient from 100% dichloromethane to dichloromethane/methanol 93:7) to give compound 6.80b (100 mg, 78%)

¹H NMR (400 MHz, 90% CDCl₃+10% MeOH-d4) δ 7.4 (d, J=1.4 Hz, 1H), 7.3-7.1 (m, 11H), 6.7 (d, J=8.9 Hz, 4H), 6.3 (dd, J=8.5, 5.5 Hz, 1H), 6.1 (td, J=6.6, 1.8 Hz, 2H), 5.1-5.0 (m, 1H), 4.97-4.87 (m, 1H), 4.30-4.23 (m, 1H), 4.2-4.0 (m, 6H), 3.9 (q, J=4.1 Hz, 1H), 3.7 (s, 6H), 3.5-3.2 (m, 7H), 2.52-2.40 (m, 2H), 2.36-2.19 (m, 3H), 2.16-2.03 (m, 1H), 1.96-1.74 (m, 4H), 1.80 (d, J=1.2 Hz, 3H), 1.78 (d, J=1.2 Hz, 3H), 1.72-1.41 (m, 6H), 1.3 (d, J=1.2 Hz, 3H), 1.23-1.08 (m, 2H), 0.7 (m, 1H).

¹³C NMR (101 MHz, 90% CDCl₃+10% MeOH-d4) δ 164.56, 164.42, 164.32, 158.73, 150.68, 150.54, 143.96, 136.15, 135.66, 135.34, 135.04, 134.97, 130.06, 128.06, 127.98, 127.25, 113.27, 111.65, 111.56, 110.97, 87.23, 85.69, 85.31, 84.73, 84.68, 84.62, 84.21, 83.07, 77.36, 75.76, 75.64, 70.26, 69.77, 68.83, 68.65, 64.90, 64.83, 64.42, 64.36, 63.08, 55.16, 39.68, 39.23, 37.82, 29.57, 29.41, 25.82, 24.44, 22.73, 22.67, 22.15, 20.76, 19.74, 19.69, 12.21, 12.12, 11.48.

³¹P NMR (162 MHz, 90% CDCl₃+10% MeOH-d4) δ 34.48, 33.77.

### Example 4. General Procedure for Synthesis of Oligomeric Compounds Containing Locked Nucleic Acid (LNA), Compound 6.89

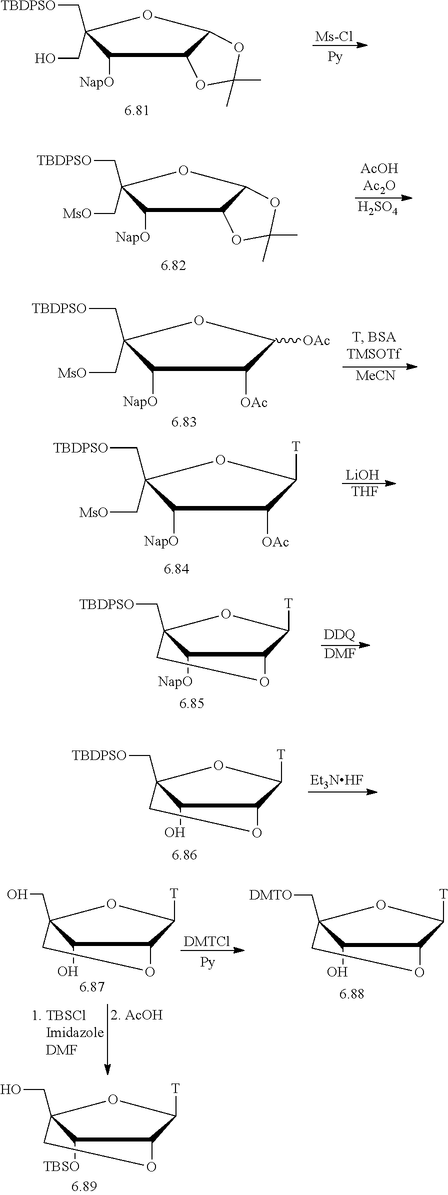

### Preparation of Compound 6.82

A solution of compound 6.81 in anhydrous pyridine is cooled in an ice bath, followed by addition of methanesulfonyl chloride. The mixture is stirred for 1 hour at room temperature, diluted with Et₂O, and washed with H₂O. The organic layer is dried, concentrated under reduced pressure, co-evaporated with toluene, and dried in vacuo to yield compound 6.82.

### Preparation of Compound 6.83

Acetic anhydride and concentrated sulfuric acid are added to a solution of compound 6.82 in acetic acid and the mixture is stirred overnight at room temperature. Water is added, and the mixture is extracted with dichloromethane. The combined organic layers are washed with saturated sodium bicarbonate solution, dried over sodium sulfate and concentrated under reduced pressure to give compound 6.83.

### Preparation of Compound 6.84

Bis(trimethylsilyl)acetamide is added to a suspension of compound 6.83 and thymine in anhydrous acetonitrile. The mixture is heated at reflux for 1 hour and cooled to room temperature. Trimethylsilyl trifluoromethanesulfonate is added dropwise, and the resulting mixture is refluxed for 3 hours. Ethyl acetate is added, and the solution is washed with saturated sodium bicarbonate solution and brine. The organic phase is dried over sodium sulfate and concentrated under reduced pressure. Purification by silica gel column chromatography affords compound 6.84.

### Preparation of Compound 6.85

Compound 6.84 was dissolved in a solution of tetrahydrofuran and methanol (2:1), and 1 M aqueous lithium hydroxide is added. After stirring for 1 hour, acetic acid is added, and the mixture is concentrated under reduced pressure. Purification by silica gel column chromatography affords compound 6.85.

### Preparation of Compound 6.86

2,3-Dichloro-5,6-dicyano-1,4-benzoquinone is added to a solution of compound 6.85 in dichloromethane and water (20:1). After stirring for 6 hours at room temperature, the reaction is concentrated under reduced pressure and dissolved in ethyl acetate. The organic layer is washed with water, 10% sodium bisulfate, saturated sodium bicarbonate, brine, dried over sodium sulfate and concentrated under reduced pressure. Purification by silica gel column chromatography affords compound 6.86.

### Preparation of Compound 6.87

Triethylamine trihydrofluoride is added to a solution compound 6.86 and anhydrous triethylamine in anhydrous tetrahydrofuran, under an atmosphere of argon. After stirring overnight, the reaction mixture is diluted with ethyl acetate and saturated sodium bicarbonate solution. The resulting mixture is stirred for 10 min. The aqueous layer is extracted with ethyl acetate. The combined organic layers are dried over sodium sulfate and concentrated under reduced pressure. Purification by silica gel column chromatography affords compound 6.87.

### Preparation of Compound 6.88

Dimethoxytrityl chloride is added to a solution of compound 6.87 in pyridine. After stirring overnight at room temperature, the reaction is quenched with methanol and the pyridine is removed under reduced pressure. The residual oil is dissolved in ethyl acetate and the organic layer is washed with saturated sodium bicarbonate solution and brine. The organic phase is dried over sodium sulfate and concentrated under reduced pressure. Purification by silica gel column chromatography affords compound 6.88.

### Preparation of Compound 6.89

tert-Butyldimethylsilyl chloride is added to a solution of compound 6.87 and imidazole in dimethylformamide. After stirring at room temperature overnight the mixture is diluted with ethyl acetate and the organic layer is washed with water, brine, dried over sodium sulfate, and concentrated under reduced pressure. Purification by column chromatography affords the desired tert-butyldimethylsilyl protected intermediate.

This intermediate is dissolved in a solution of acetic acid and water (2:1), and stirred at room temperature. Ethyl acetate and water are added to the reaction mixture. The organic layer is isolated and washed with saturated sodium bicarbonate solution, brine, dried over sodium sulfate and concentrated under reduced pressure. Purification by silica gel column chromatography affords compound 6.89.

### Example 5. T_{M} Evaluation of Constrained Backbone Oligomeric Compounds

Tₘ for the binding of oligonucleotides described below to their RNA complements was evaluated and is reported in the table below. Surface plasmon resonance was used to analyze the on rate and off-rates of the binding of the oligonucleotides in the tables below to their respective RNA complement, and a K_{d} was calculated based on these results and is reported below.

The sequence for the modified oligonucleotides in the table below is GGATGTTTCTCGA. Each internucleoside linkage is a phosphodiester internucleoside linkage except for the two allyl phosphonate modified internucleoside linkages indicated in the structure below. Each nucleoside comprises a 2′-β-D-deoxyribosyl sugar moiety.

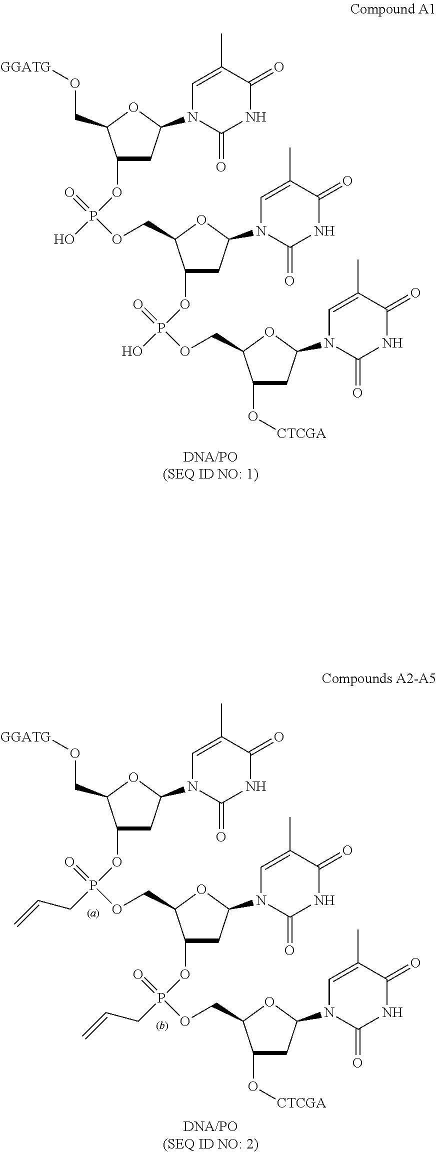

TABLE 1

| | | P config. | T_{M} | K_{d} | Kₒₙ | K_{off} | % Duplex @ |
|---|---|---|---|---|---|---|---|
| Compound | n | (a, b) | (° C.) | (nM) | (M⁻¹S⁻¹) | (S⁻¹) | 20 nM |
| A1 | — | — | 49.5 | 0.26 | 4.44 × 10⁵ | 11.3 × 10⁻⁵ | 100 |
| A2 | — | (S, R) | 42.8 | 1.35 | 6.04 × 10⁵ | 81.5 × 10⁻⁵ | 100 |
| A3 | — | (S, S) | 38.1 | 13.8 | 1.96 × 10⁵ | 271 × 10⁻⁵ | N.D. |
| A4 | — | (R, S) | 42.4 | 1.19 | 6.60 × 10⁵ | 78.8 × 10⁻⁵ | N.D. |
| A5 | — | (R, R) | 46.9 | 0.392 | 5.17 × 10⁵ | 20.3 × 10⁻⁵ | 100 |

These results indicate that compounds comprising two allyl phosphonate internucleoside linkages are less stable than the parent oligonucleotide comprising a full phosphodiester backbone.

The sequence for the modified oligonucleotides in the table below is GGATGTTTCTCGA. Each internucleoside linkage is a phosphodiester internucleoside linkage except for the two backbone constrained internucleoside linkages that form a macrocycle, as indicated in the structure below. Each nucleoside comprises a 2′-β-D-deoxyribosyl sugar moiety. The size of the macrocyclic ring formed varies with (n), from 11 atoms when n=1 to 15 atoms when n=5, and the stereochemistry at the phosphates (a) and (b) is also reported in the table below.

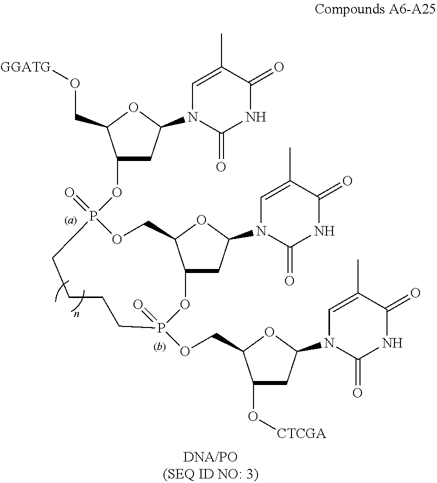

TABLE 2

| | | P config. | T_{M} | K_{d} | Kₒₙ | K_{off} | % Duplex @ |
|---|---|---|---|---|---|---|---|
| Compound | n | (a, b) | (° C.) | (nM) | (M⁻¹S⁻¹) | (S⁻¹) | 20 nM |
| A6 | 1 | (S, R) | 34.4 | N.D. | N.D. | N.D. | 0 |
| A7 | 1 | (S, S) | 31.7 | N.D. | N.D. | N.D. | 0 |
| A8 | 1 | (R, S) | 33.7 | N.D. | N.D. | N.D. | 0 |
| A9 | 1 | (R, R) | 30.3 | N.D. | N.D. | N.D. | 0 |
| A10 | 2 | (S, R) | 38.9 | 7.620 | 9.91 × 10⁵ | 7.55 × 10⁻³ | 55 |
| A11 | 2 | (S, S) | 39.0 | 13.7 | 1.33 × 10⁶ | 1.82 − 2 | 43 |
| A12 | 2 | (R, S) | 41.9 | 1.93 | 1.01 × 10⁶ | 1.95 × 10⁻³ | 74 |
| A13 | 2 | (R, R) | 35.8 | 13.97 | 3.10 × 10⁵ | 4.33 × 10⁻³ | 32 |
| A14 | 3 | (S, R) | 40.1 | 4.480 | 1.39 × 10⁶ | 6.24 × 10⁻³ | 63 |
| A15 | 3 | (S, S) | 45.1 | 0.74 | 8.64 × 10⁵ | 6.42 × 10⁻⁴ | 100 |
| A16 | 3 | (R, S) | 39.6 | 7.30 | 1.72 × 10⁶ | 1.26 × 10⁻² | 58 |
| A17 | 3 | (R, R) | 37.7 | 23.350 | 4.58 × 10⁵ | 1.07 × 10⁻² | 23 |
| A18 | 4 | (S, R) | 46.1 | 0.440 | 5.75 × 10⁵ | 2.53 × 10⁻⁴ | 100 |
| A19 | 4 | (S, S) | 42.7 | 1.03 | 7.61 × 10⁵ | 7.84 × 10⁻⁴ | N.D. |
| A20 | 4 | (R, S) | 46.2 | 0.251 | 7.05 × 10⁵ | 1.77 × 10⁻⁴ | N.D. |
| A21 | 4 | (R, R) | 41.6 | 2.340 | 8.47 × 10⁵ | 1.98 × 10⁻³ | 78 |
| A22 | 5 | (S, R) | 48.4 | 0.338 | 5.36 × 10⁵ | 18.1 × 10⁻⁵ | N.D. |
| A23 | 5 | (R, S) | 46.4 | 0.161 | 18.90 × 10⁵ | 412 × 10⁻⁵ | N.D. |
| A24 | 5 | (S, S) | 41.1 | 2.180 | 7.12 × 10⁵ | 11.4 × 10⁻⁵ | N.D. |
| A25 | 5 | (R, R) | 42.4 | 0.70 | 2.29 × 10⁵ | 16.10 × 10⁻⁵ | N.D. |

These results indicate that smaller macrocycles (n=1-2; ring size of 11 or 12) are highly destabilizing.

The sequence for the modified oligonucleotides in the table below is GGATGTTTCTCGA. Each internucleoside linkage is a phosphodiester internucleoside linkage except for the two backbone constrained internucleoside linkages that form a macrocycle, as indicated in the structure below. Each nucleoside comprises a 2′-β-D-deoxyribosyl sugar moiety. The macrocycle contains 15 atoms, and the stereochemistry at the phosphates (a) and (b) is also reported in the table below.

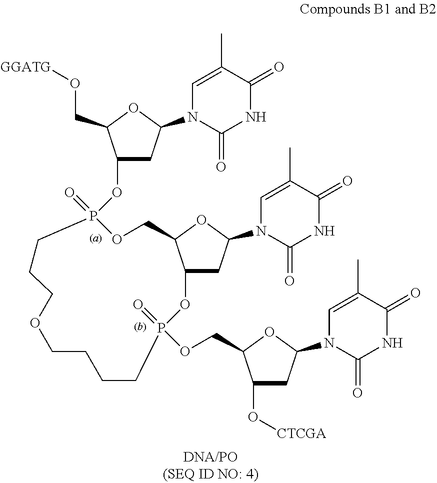

TABLE 3

| | | P config. | T_{M} | K_{d} | % Duplex |
|---|---|---|---|---|---|
| Compound | n | (a, b) | (° C.) | (nM) | @ 20 nM |
| B1 | — | (S, R) | 47.5 | 0.168 | N.D. |
| B2 | — | (R, R) | 42.2 | 7.020 | N.D. |

1. A compound comprising the formula: 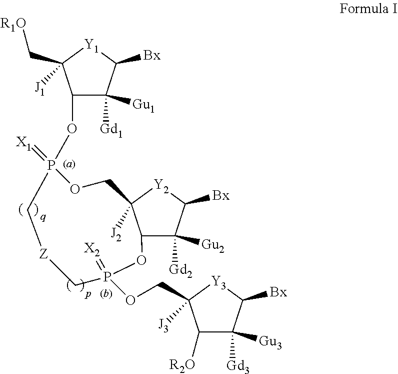
wherein each Bx is, independently, a heterocyclic base moiety;
R₁ is H, a hydroxyl protecting group or a conjugate group;
R₂ is H, a hydroxyl protecting group, a conjugate group, or a reactive phosphorous group;
(a) and (b) are the stereochemistry at the phosphate and are independently selected from (R), (S), and (R,S);
p is from 1 to 7;
q is from 1 to 7;
and wherein p+q is greater than or equal to 4;
Z is CH₂ or O;
each X is, independently, O or S;
each Y is, independently, O or S;
either J₁ and G_{d1} form a J₁ and G_{d1} bridge and Gᵤ₁ is H, or J₁ is H and G_{d1} and Gᵤ₁ are independently selected from H, OH, halogen or O—[C(R₃)(R₄)]ₙ—[(C═O)ₘ—X^{G}]ⱼ—R₅;
either J₂ and G_{d2} form a J₂ and G_{d2} bridge and Gᵤ₁ is H, or J₂ is H and G_{d2} and Gᵤ₂ are independently selected from H, OH, halogen or O—[C(R₃)(R₄)]ₙ—[(C═O)ₘ—X^{G}]ⱼ—R₅;
either J₃ and G_{d3} form a J₃ and G_{d3} bridge and Gᵤ₁ is H, or J₃ is H and G_{d3} and Gᵤ₃ are independently selected from H, OH, halogen or O—[C(R₃)(R₄)]ₙ—[(C═O)ₘ—X^{G}]ⱼ—R₅;
wherein each J to G_{d} bridge has a formula independently selected from —CH(CH₃)—O— or —(CH₂)ₖ—O—, wherein k is from 1 to 3;
each R₃ and R₄ is, independently, H, halogen, C₁-C₆ alkyl or substituted C₁-C₆ alkyl;
each X^{G} is O, S or N(E₁);
R₅ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(═X₂)J₁, OC(═X₂)N(J₁)(J₂) and C(=Q₂)N(J₁)(J₂);
Q₂ is O, S or NJ₃;
each J₁, J₂ and J₃ is, independently, H or C₁-C₆ alkyl.

2. The compound of claim 1, wherein each Y is O.

3. The compound of claim 1, wherein each X is S.

4. The compound of claim 1, wherein each X is O.

5. The compound of claim 1, wherein p+q equals 6, 7, or 8.

6.-8. (canceled)

9. The compound of claim 1, wherein R₁ and R₂ are independently, hydrogen or a hydroxyl protecting group.

10. The compound of claim 1, wherein R₁ is a hydroxyl protecting group and R₂ is a reactive phosphorous group.

11. The compound of claim 1, wherein each Gᵤ is H, and each G_{d} is independently selected from H, O-methoxyethyl, O-methyl, or fluoro.

12. (canceled)

13. The compound of claim 1, wherein at least one J forms a bridge with at least one G_{d}.

14. The compound of claim 13, wherein the bridge has the formula —(CH₂)—O—.

15.-21. (canceled)

22. An oligomeric compound comprising a modified oligonucleotide consisting of 8 to 40 linked nucleosides, wherein the modified oligonucleotide comprises a region having the formula: 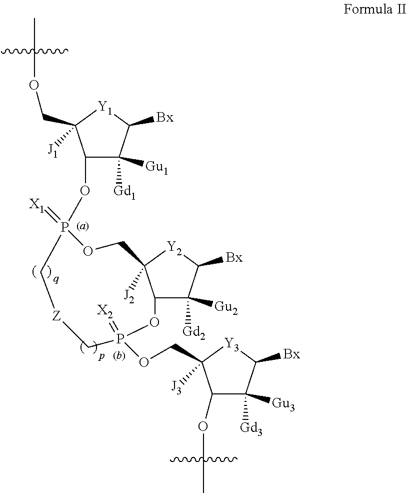
wherein each Bx is, independently, a heterocyclic base moiety;
(a) and (b) are the stereochemistry at the phosphate and are independently selected from (R), (S), and (R,S);
p is from 1 to 7;
q is from 1 to 7;
and wherein p+q is greater than or equal to 4;
Z is CH₂ or 0;
each X is, independently, O or S;
each Y is, independently, O or S;
either J₁ and G_{d1} form a J₁ and G_{d1} bridge and Gᵤ₁ is H, or J₁ is H and G_{d1} and Gᵤ₁ are independently selected from H, OH, halogen or O—[C(R₃)(R₄)]ₙ—[(C═O)ₘ—X^{G}]ⱼ—R₅;
either J₂ and G_{d2} form a J₂ and G_{d2} bridge and Gᵤ₁ is H, or J₂ is H and G_{d2} and Gᵤ₂ are independently selected from H, OH, halogen or O—[C(R₃)(R₄)]ₙ—[(C═O)ₘ—X^{G}]ⱼ—R₅;
either J₃ and G_{d3} form a J₃ and Gas bridge and Gᵤ₁ is H, or J₃ is H and Gas and Gᵤ₃ are independently selected from H, OH, halogen or O—[C(R₃)(R₄)]ₙ—[(C═O)ₘ—X^{G}]ⱼ—R₅;
wherein each J to G_{d} bridge has a formula independently selected from —CH(CH₃)—O— or —(CH₂)ₖ—O—, wherein k is from 1 to 3;
each R₃ and R₄ is, independently, H, halogen, C₁-C₆ alkyl or substituted C₁-C₆ alkyl;
each X^{G} is O, S or N(E₁);
R₅ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(═X₂)J₁, OC(═X₂)N(J₁)(J₂) and C(=Q₂)N(J₁)(J₂);
Q₂ is O, S or NJ₃;
each J₁, J₂ and J₃ is, independently, H or C₁-C₆ alkyl.

23. The oligomeric compound of claim 22, wherein each Y is O.

24. The oligomeric compound of claim 22, wherein each X is S.

25. The oligomeric compound of claim 22, wherein each X is O.

26. The oligomeric compound of any of claims 22-25, wherein p+q equals 6, 7, or 8.

27.-29. (canceled)

30. The oligomeric compound of claim 22, wherein each Gᵤ is H, and each G_{d} is independently selected from H, O-methoxyethyl, O-methyl, or fluoro.

31. (canceled)

32. The oligomeric compound of claim 22, wherein at least one J forms a bridge with at least one G_{d}.

33. The oligomeric compound of claim 32, wherein the bridge has the formula —(CH₂)—O—.

34.-40. (canceled)

41. The oligomeric compound of claim 22, wherein the modified oligonucleotide consists of 12-24, 12-20, 16-20, 18-20, or 22-23 linked nucleosides.

42.-53. (canceled)

54. The oligomeric compound of claim 22, wherein the modified oligonucleotide has a sugar motif comprising:
a 5′-region consisting of 1-6 linked 5′-region nucleosides;
a central region consisting of 6-10 linked central region nucleosides; and
a 3′-region consisting of 1-6 linked 3′-region nucleosides; wherein
each of the 5′-region nucleosides and each of the 3′-region nucleosides comprises a modified sugar moiety and each of the central region nucleosides comprises a 2′-β-D-deoxyribosyl sugar moiety.

55.-63. (canceled)


SEQUENCE LISTING (4 sequences)

SEQ ID NO: 1 - DNA, 13 bp, synthetic construct
  source 1..13: mol_type = other DNA
ggatgtttctcga

SEQ ID NO: 2 - DNA, 13 bp, synthetic construct
  source 1..13: mol_type = other DNA
  modified_base 6: mod_base = OTHER; note = 2'-deoxythymidine 3'-allylphosphonate
  modified_base 7: mod_base = OTHER; note = 2'-deoxythymidine 3'-allylphosphonate
ggatgtttctcga

SEQ ID NO: 3 - DNA, 13 bp, synthetic construct
  source 1..13: mol_type = other DNA
  modified_base 6: mod_base = OTHER; note = 2'-deoxythymidine 3'-alkylphosphonate bridge to nt 7
  modified_base 7: mod_base = OTHER; note = 2'-deoxythymidine 3'-alkylphosphonate bridge to nt 6
ggatgtttctcga

SEQ ID NO: 4 - DNA, 13 bp, synthetic construct
  source 1..13: mol_type = other DNA
  modified_base 6: mod_base = OTHER; note = 2'-deoxythymidine 3'-propoxybutylphosphonate bridge to nt 7
  modified_base 7: mod_base = OTHER; note = 2'-deoxythymidine 3'-propoxybutylphosphonate bridge to nt 6
ggatgtttctcga